(19) United States
(12) Patent Application Publication
(10) Pub. No.: US 2016/0060612 A1
(43) Pub. Date: Mar. 3, 2016

(54) Method of Isolating and Purifying Fusion Protein Comprising Factor VII

(71) Applicant: SK CHEMICALS CO., LTD., Seongnam-si, Gyeonggi-do (KR)

(72) Inventors: Ji-Hye Lee, Gyeonggi-do (KR); Seok-chan Kang, Gyeonggi-do (KR); Yangkyun Ryu, Seoul (KR); Ho Soon Lee, Gyeonggi-do (KR); In-Young Song, Seoul (KR); Hun-Taek Kim, Seoul (KR)

(21) Appl. No.: 14/770,262

(22) PCT Filed: Feb. 25, 2014
(86) PCT No.: PCT/KR2014/001526

§ 371 Date: Aug. 25, 2015

(30) Foreign Application Priority Data
Feb. 25, 2013 (KR) 10-2013-0019913

(51) Int. Cl. C12N 9/64; C07K 1/22; C07K 1/18; C07K 14/79

(52) U.S. Cl. CPC C12N 9/6437; C07K 14/79; C07K 1/22; C07K 1/18; C07K 2319/50; C12Y 304/21021

(57) ABSTRACT

The present invention provides a method of isolating and purifying a fusion protein comprising factor VII, and more specifically relates to a method of isolating and purifying a fusion protein comprising factor VII and transferrin, to a high degree of purity. Because the present invention provides a method whereby a recombinant fusion protein comprising factor VII can be isolated and purified to a high degree of purity, the invention is useful in producing a pharmaceutical preparation comprising factor VII that can be used in situations in which copious bleeding occurs such as surgery.

### TECHNICAL FIELD

The present invention relates to a method of isolating and purifying a fusion protein containing factor VII (FVII), and more particularly, to a method of isolating and purifying a fusion protein including FVII and transferrin to a high degree of purity.

### BACKGROUND ART

Blood coagulation factor VII (hereinafter referred to as ‘factor VII’ or ‘FVII’) is a plasma protein which is dependent on vitamin K, and it plays a key role in the initiation of blood coagulation. In blood and tissues, over 50 substances are involved in the blood coagulation. In this case, the substances taking part in the blood coagulation are referred to as procoagulants, and the substances preventing the blood coagulation are referred to as anticoagulants. Two conflicting groups of the substances in blood and tissues are always in equilibrium, but the anticoagulants are generally predominant to prevent blood from coagulating. However, when blood vessels are damaged, the procoagulants become predominant over the anticoagulants in this affected part. In this case, prothrombin, which is one of the plasma proteins in blood produced in the liver by the action of vitamin K, is converted into thrombin by the action of calcium ions or platelets. Then, the thrombin functions to convert fibrinogen to fibrin, which leads to coagulation of blood.

Depending on activation mechanisms, pathways for the blood coagulation are divided into an extrinsic pathway starting from the damage of blood vessel walls or their surrounding tissues and an intrinsic pathway starting from blood itself. Between these two pathways, the extrinsic pathway is initiated as tissue factors (TFs) present in cell membranes are exposed to circulating blood, when blood vessel walls are damaged, thereby to form a complex with FVII or active factor VII (FVIIa: present in blood at an amount corresponding to approximately 1% of the total mass of FVII proteins) present in blood. Since such a complex has catalytic activities, it acts on factor X (FX) present on the cell surface so that the FX is converted into activated factor X (FXa). Then, the FXa results in a blood coagulation cascade to convert factor IX (FIX) into active factor IX (FIXa).

FVII is a single-chain glycoprotein which consists of 406 amino acids, has a molecular weight of approximately 50 kDa, and is secreted by the liver into the blood stream as inert zymogen. FVII contains four domains: an amino-terminal-carboxyglutamate (Glu) domain, two epidermal growth factor (EGF)-like domains, and a serine protease domain (Hagen F S et al., Proc. Natl. Acad. Sci., USA, 83(8): 2412-2416, 1986). FVII is converted into its active form FVIIa by the proteolysis of a single peptide bond at Arg152-Ile153, leading to the formation of two polypeptide chains, an N-terminal light chain (24 kDa) and a C-terminal heavy chain (28 kDa), which are linked to each other by a disulfide bond. Factor VII is present in the plasma at a concentration of 500 ng/mL. 1% (i.e., 5 ng/mL) of the Factor VII is present as Factor VIIa.

FVII has the shortest plasma half-life among the protein in the serum, and thus it is a problem that the FVII should be repeatedly administered so as to effectively stop the bleeding. To solve the above problems, techniques capable of enhancing the half-life of FVII to improve the patients' convenience and to enhance the efficiency, for example, PEGylation, carboxyl terminal peptide (CTP), and albumin fusion techniques, have been studied so far.

Particularly, the present inventors have proposed a type of a fusion protein in which FVII is fused to transferrin as a way to enhance the in vivo half-life of FVII (see Korean Unexamined Patent Publication No. 10-2011-0133454). Since the FVII-transferrin fusion protein has a longer half-life in vivo than wild-type FVII and also retains biological activities of FVII, it may be able to replace the use of FVII for the treatment.

The fusion protein may be expressed in host cells (e.g., CHO cells, etc.) using a recombinant vector having a DNA sequence coding for the fusion protein inserted thereto. However, a method of isolating and purifying the expressed protein remains to be established. In the prior art, methods of isolating and purifying a blood coagulation factor itself using column chromatography were studied, but the purity of the isolated and purified protein was very low, which makes it difficult to apply them to the isolation and purification of the fusion protein.

### DISCLOSURE

### Technical Problem

Therefore, the present invention is designed to solve the problems of the prior art, and therefore it is an object of the present invention to provide a method of isolating and purifying a fusion protein, which includes factor VII expressed by a genetic engineering methods, to a high degree of purity.

### Technical Solution

A method of isolating and purifying a fusion protein containing factor VII is provide, which comprises 1) isolating and purifying the fusion protein containing factor VII expressed in animal cells by affinity chromatography using a resin having a structure represented by Formula 1 as a stationary phase or by mixed-mode chromatography using a ceramic fluoroapatite gel (Ca₁₀(PO₄)₆F₂) as a stationary phase, and 2) further isolating and purifying the fusion protein by anion exchange chromatography using Q-sepharose FF gel as a stationary phase.

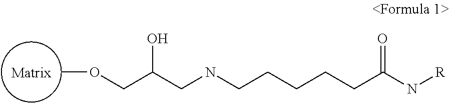

In Formula 1, the matrix is cross-linked agarose, and R is a factor VII binding protein serving as a ligand.

### Advantageous Effects

Because the present invention provides a method capable of isolating and purifying a recombinant fusion protein containing factor VII to a high degree of purity, the method according to one exemplary embodiment of the invention can be useful in manufacturing a pharmaceutical preparation including the factor VII that can be used in cases in which copious bleeding occurs such as surgery.

### DESCRIPTION OF DRAWINGS

FIG. 1 is a schematic diagram showing a cloning procedure for constructing a FVII-Tf expression vector from a vector containing a cDNA coding for a FVII and a vector containing a cDNA coding for a transferrin (Tf).

FIG. 2 is a schematic diagram showing a process of preparing a FVII-GS1 (linker)-Tf expression vector using an overlapping polymerase chain reaction (PCR).

FIG. 3 is a schematic diagram showing a process of preparing a FVII-GS linker-Tf expression vector containing GS3, GS5, GS7, GS9, GS11, GS13, GS15, or GS-1-T as a linker.

FIG. 4 shows the results obtained by confirming the presence of FVII-Tf, FVII-GS1-Tf, FVII-GS3-Tf, FVII-GS5-Tf, FVII-GS7-Tf, FVII-GS9-Tf, FVII-GS11-Tf, FVII-GS13-Tf, FVII-GS15-Tf, FVII-GS1-T-Tf, FVII-Helix-Tf, which are the fusion proteins according to one exemplary embodiment of the present invention, and FVII (NovoSeven™) by Western blotting.

FIG. 5 is a graph showing the specific activities of FVII-Tf, FVII-GS1-Tf, FVII-GS3-Tf, FVII-GS5-Tf, FVII-GS7-Tf, FVII-GS9-Tf, FVII-GS11-Tf, FVII-GS13-Tf, FVII-GS15-Tf, FVII-GS1-T-Tf, and FVII-Helix-Tf, which are the fusion proteins according to one exemplary embodiment of the present invention.

FIG. 6 is a diagram showing a structure of the fusion protein, FVII-GS1-T-Tf, according to one exemplary embodiment of the present invention, which includes a linker and a restriction endonuclease recognition sequences flanking at both ends thereof.

FIG. 7 is a diagram showing the Western blotting results of FVII-Tf, FVII-GS1-Tf, FVII-GS1-T-Tf, FVII-GS3-Tf, FVII-GS15-Tf, which are the purified fusion proteins according to one exemplary embodiment of the present invention, NovoSeven®, and FVII.

FIG. 8 is a chromatogram showing the light absorbance measured at a UV wavelength of 280 nm for the recombinant FVII fusion protein (FVII-GS1-T-Tf) according to one exemplary embodiment of the present invention after the recombinant FVII fusion protein (FVII-GS1-T-Tf) is isolated and purified under the elution conditions described in Example <9-2> by affinity chromatography using an XK16/20 VIISelect as a stationary phase.

FIG. 9 shows the results analyzed for the recombinant FVII fusion protein (FVII-GS1-T-Tf) according to one exemplary embodiment of the present invention by SDS-PAGE after the recombinant FVII fusion protein (FVII-GS1-T-Tf) is isolated and purified under the elution conditions described in Example <9-2> by affinity chromatography using an XK16/20 VIISelect as a stationary phase.

FIG. 10 is a chromatogram showing the light absorbance measured at a UV wavelength of 280 nm for the recombinant FVII fusion protein (FVII-GS1-T-Tf) according to one exemplary embodiment of the present invention after the recombinant FVII fusion protein (FVII-GS1-T-Tf) is isolated and purified under the elution conditions described in Example <9-2> by affinity chromatography using an XK16/20 VIISelect as a stationary phase and further isolated and purified by anion exchange chromatography using Q-sepharose FF as a stationary phase.

FIG. 11 shows the results analyzed for the recombinant FVII fusion protein (FVII-GS1-T-Tf) according to one exemplary embodiment of the present invention by SDS-PAGE after the recombinant FVII fusion protein (FVII-GS1-T-Tf) is isolated and purified under the elution conditions described in Example <9-2> by affinity chromatography using an XK16/20 VIISelect as a stationary phase and further isolated and purified by anion exchange chromatography using Q-sepharose FF as a stationary phase.

FIG. 12 is a chromatogram showing the light absorbance measured at a UV wavelength of 280 nm for the recombinant FVII fusion protein (FVII-GS1-T-Tf) according to one exemplary embodiment of the present invention after the recombinant FVII fusion protein (FVII-GS1-T-Tf) is isolated and purified under the elution conditions described in Comparative Example 1 by affinity chromatography using an XK16/20 VIISelect as a stationary phase.

FIG. 13 is a chromatogram showing the light absorbance measured at a UV wavelength of 280 nm for the recombinant FVII fusion protein (FVII-GS1-T-Tf) according to one exemplary embodiment of the present invention after the recombinant FVII fusion protein (FVII-GS1-T-Tf) is isolated and purified by mixed-mode chromatography using XK16/20 ceramic fluoroapatite as a stationary phase.

FIG. 14 shows the results analyzed for the recombinant FVII fusion protein (FVII-GS1-T-Tf) according to one exemplary embodiment of the present invention by SDS-PAGE after the recombinant FVII fusion protein (FVII-GS1-T-Tf) is isolated and purified by mixed-mode chromatography using XK16/20 ceramic fluoroapatite as a stationary phase

FIG. 15 is a chromatogram showing the light absorbance measured at a UV wavelength of 280 nm for the recombinant FVII fusion protein (FVII-GS1-T-Tf) according to the present invention after the recombinant FVII fusion protein (FVII-GS1-T-Tf) is isolated and purified by mixed-mode chromatography using XK16/20 ceramic hydroxyapatite as a stationary phase.

FIG. 16 shows the results analyzed for the recombinant FVII fusion protein (FVII-GS1-T-Tf) according to one exemplary embodiment of the present invention by SDS-PAGE after the recombinant FVII fusion protein (FVII-GS1-T-Tf) is isolated and purified by mixed-mode chromatography using XK16/20 ceramic hydroxyapatite as a stationary phase.

### BEST MODE

The present invention provides a method of isolating and purifying a fusion protein containing factor VII, which includes 1) isolating and purifying a fusion protein containing factor VII expressed in animal cells by affinity chromatography using a resin having a structure represented by Formula 1 as a stationary phase, or mixed-mode chromatography using a ceramic fluoroapatite gel (Ca₁₀(PO₄)₆F₂) as a stationary phase, and 2) further isolating and purifying the fusion protein by anion exchange chromatography using Q-sepharose FF gel as a stationary phase.

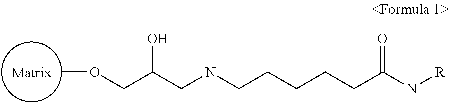

In Formula 1, the matrix is cross-linked agarose, and R is a factor VII binding protein serving as a ligand.

In this specification, the phase “fusion protein containing factor VII” refers to a fusion protein in which blood coagulation factor VII (FVII) is linked to any fusion partner, which is a fusion protein in which the activity of factor VII for blood coagulation, which is not lost or alleviated by the fusion partner.

Examples of the fusion protein includes a fusion protein containing factor VII and transferrin, a fusion protein containing factor VII and albumin, a fusion protein containing factor VII and fibrinogen, a fusion protein containing factor VII and IgA, a fusion protein containing factor VII and IgM, or a fusion protein containing factor VII and a Fc domain of an antibody, but the present invention is not limited thereto.

According to one exemplary embodiment, the fusion protein that may be used in the method of the present invention is a fusion protein containing factor VII (FVII) and transferrin (Tf). The FVII and transferrin residues of the FVII-Tf fusion protein may be derived from any mammal, preferably human FVII and transferrin. More preferably, the FVII residue and transferrin have a sequence homology of 95% or more to respective wild-type proteins found in human blood. Most preferably, FVII has an amino acid sequence set forth in SEQ ID NO: 1, and transferrin has an amino acid sequence set forth in SEQ ID NO: 2.

Also, the FVII-Tf fusion protein includes functional equivalents or functional derivatives having substantially the same functional activities. Examples of such functional equivalents may include variants mutated by any of deletions, insertions, non-conservative or conservative substitutions, or combinations thereof in any amino acid residue in the respective amino acid sequences set forth in SEQ ID NOs: 1 and 2. In this case, such modifications do not substantially alter the active sites or domains which give the biological activities of FVII. Occasionally, the FVII-Tf fusion protein may be altered to enhance or reduce their physical and chemical properties, and it may be modified by phosphorylation, sulfation, acrylation, glycosylation, methylation, farnesylation, acetylation, amidation, etc. As long as the activities of FVII are substantially retained by such modifications, such functional derivatives are also encompassed in the scope of the present invention.

The FVII-Tf fusion protein is preferably a fusion protein in which transferrin is linked to the C-terminus of FVII. Since the N-terminus of FVII is exposed in the fusion protein having FVII and transferrin linked in the order listed herein, the fusion protein has a superior effect as a therapeutic agent, compared to the fusion protein having transferrin and FVII linked in the order listed herein (see Table 3).

Also, the FVII-Tf fusion protein may include a restriction endonuclease recognition sequence between FVII and transferrin in order to promote the insertion of a linker to be described below. Various restriction endonuclease recognition sequences known in the related art may be used as the restriction endonuclease recognition sequence. Preferably, the restriction endonuclease recognition sequence may contain an AgeI recognition sequence (A/CCGGT). That is, the fusion proteins in which the restriction endonuclease recognition sequence is linked to the C-terminus of FVII and transferrin is linked to the restriction endonuclease recognition sequence is encompassed in the scope of the present invention.

In addition, the FVII-Tf fusion protein may include a linker between the FVII residue and the transferrin residue. The linker may have 1 to 100 amino acids, preferably 1 to 75 amino acids, and more preferably 5 to 25 amino acids. Here, any peptides may be used as long as they can separate the FVII residue and the transferrin residue. The linker may have a stable secondary structure such as a double helix structure, or it may be derived from an IgG hinge region. Preferably, since the linker is freely rotatable in an aqueous solution and does not have a fixed structure, the linker is not immunogenic and minimizes the potential interference of two fusion partners to enhance the FVII activities of the fusion protein. As such a linker, the linker may be a helix linker having an amino acid sequence set forth in SEQ ID NO: 11. Also, such a flexible linker may include glycine (G) and serine (S) in a repeated or random pattern. Examples of the linker include (GGGGS)_{N} (N is an integer ranging from 1 to 20). Preferably, the linker has an amino acid sequence set forth in one of SEQ ID NO: 3 to 10 (see Table 1). Also, an amino acid sequence having homology of 80% or more, preferably having homology of 85% or more to the amino acid sequence of the linker may also be used in the FVII-Tf fusion protein.

Also, the linker may include a protease digestion site recognizable by proteases plentifully present in damaged tissues. The digestion site may be a site cleaved by at least one protease selected from the group consisting of thrombin, factor Xa, factor IXa, and factor VIIa. The fusion protein including such a protease digestion site is cleaved into respective proteins of FVII and transferrin at the working site. Then, the FVII and transferrin serve as the respective proteins. Preferably, the linker has an amino acid sequence set forth in SEQ ID NO: 12 (see Table 1).

The linker may be more readily inserted via the restriction endonuclease recognition sequence inserted between FVII and transferrin. Therefore, the restriction endonuclease recognition sequence(s) may be present at either of upstream or downstream end or both ends of the linker, and it may be translated into amino acids corresponding to the n.t sequence. For example, when an AgeI restriction endonuclease recognition sequence is used, Thr may be present upstream of the linker, and Thr-Gly may be present downstream of the linker. That is, when a linker such as (GGGGS)3 is used, the linker is present in the form of -T(GGGGS)₃TG-. An amino acid(s) translated at the upstream or downstream of the linker may vary according to the restriction endonuclease recognition sequence used herein, but the activities of the fusion protein are not changed due to the presence of the translated amino acid(s) (see Table 5).

The FVII-Tf fusion protein has FVII specific activity of 0.7 or more, compared to non-fused wild-type FVII. For example, the fusion protein including the FVII having the amino acid sequence set forth in SEQ ID NO: 1 and the transferrin having the amino acid sequence set forth in SEQ ID NO: 2 has FVII specific activity of approximately 0.82 to 0.92, compared to the non-fused wild-type FVII (see Tables 2 and 3). For example, the fusion protein including the FVII having the amino acid sequence set forth in SEQ ID NO: 1, the linker having the amino acid sequence set forth in SEQ ID NO: 3, and the transferrin having the amino acid sequence set forth in SEQ ID NO: 2 also has FVII specific activity of approximately 0.97, compared to the non-fused wild-type FVII (see Table 2). In addition, a fusion protein having another linker inserted between FVII and transferrin also has FVII specific activity of approximately 0.74 to 1, compared to the non-fused wild-type FVII (see Table 2). Further, the FVII-Tf fusion protein has a half-life 3 to 4 times longer than the FVII not linked with transferrin (see Table 6).

In the present invention, the fusion protein containing factor VII may be expressed by introducing a DNA sequence coding for the fusion protein into host cells.

The DNA sequence coding for the fusion protein may be widely altered or modified within a range in which the amino acid sequence of the fusion protein is not altered, in consideration of the codon degeneracy or codons preferred in organisms in which the fusion protein is expressed. For example, in the case of the FVII-Tf fusion protein, the DNA sequence coding for the FVII-Tf fusion protein is preferably a DNA sequence having the nucleotide sequence set forth in one of SEQ ID NOs: 13 to 24.

The DNA sequence coding for the fusion protein may be introduced into host cells using a recombinant expression vector. In the present invention, the term “vector” refers to a means for introducing a DNA sequence coding for the fusion protein into host cells and expressing the fusion protein, and the term includes all types of conventional vectors including a plasmid vector, a cosmid vector, a bacteriophage vector, and a viral vector, with a plasmid vector being preferred. Proper expression vectors include n.t sequences coding for a signal peptide or a leader peptidee for membrane targeting or secretion as well as expression control elements such as a promoter, an initiation codon, a stop codon, a polyadenylation signal, and an enhancer, and they may be variously prepared according to a purpose of use. The initiation codon and the stop codon should necessarily act in a subject when a gene construct is administered to the subject, and they should be fused in-frame with a coding sequence. Also, the expression vector includes a selectable marker for selecting host cells containing the vector, and a replicable expression vector includes a replication origin. The vector may be self-replicated or integrated into a host cell DNA sequence. Specifically, the recombinant expression vector according to one exemplary embodiment of the present invention may be constructed by inserting a DNA sequence coding for the fusion protein into pcDNA3.1-hygro vector.

In the present invention, conventional animal cells known in the related art may be used as the host cells used to express the fusion protein. Examples of the host cells may include Chinese hamster ovary (CHO) cells, human embryonic kidney cells (HEK293), hamster kidney cells (BHK 21), human liver cancer cells (Hep G2), and the like, but the present invention is not limited thereto.

Methods known in the related art may be used to transform host cells with the recombinant expression vector according to one exemplary embodiment of the present invention. Such methods include electroporation, plasmogamy, calcium phosphate (CaPO₄) precipitation, and calcium chloride (CaCl₂) precipitation, but the present invention is not limited thereto.

The present invention is characterized in that the fusion protein is isolated and purified through the steps of 1) isolating and purifying a fusion protein containing factor VII expressed in animal cells by affinity chromatography using a resin having the above-described structure as a stationary phase, or mixed-mode chromatography using a ceramic fluoroapatite gel (Ca₁₀(PO₄)₆F₂) as a stationary phase, and 2) further isolating and purifying the fusion protein by anion exchange chromatography using a Q-sepharose FF gel as a stationary phase.

Prior to performing the chromatography, the fusion protein according to one exemplary embodiment of the present invention may selectively undergo the following steps:

a) culturing host cells transformed with a recombinant expression vector and isolating the cells from culture medium; and

b) concentrating the cell culture medium by ultrafiltration.

In step a), the host cells are cultured according to culture methods widely known in the related art. Next, the culture is centrifuged to isolate the cells from the culture medium, and the isolated culture medium is applied to step b). The efficiency of ultrafiltration or dialysis in step b) may be enhanced through the centrifugation in step a).

In step b), the isolated cell culture medium may be preferably concentrated by ultrafiltration (UF) using a membrane having a cut-off value (i.e., a retention time) of 30 kDa. The membrane for ultrafiltration may vary according to the characteristics of a target protein. Generally, the target protein may be enriched without any great loss by passing the cell culture medium through a membrane having a smaller pore size than the molecular weight of the target protein. For example, a FVII fusion protein having a molecular weight of 130 kDa may be enriched by passing the cell culture medium through a membrane having a pore size of 30 kDa. After the ultrafiltration, a buffer used in this step may also be replaced with an equilibration buffer used in subsequent procedures. An exemplary equilibration buffer may a buffer (pH 6.5) including 25 mM imidazole and 0.02% Tween 80.

According to one exemplary embodiment, the concentrate having undergone the above the concentration procedure is isolated and purified by affinity chromatography using a resin having a structure represented by the following Formula 1 as a stationary phase.

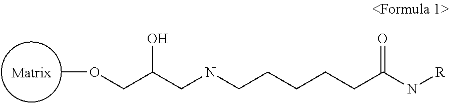

The resin used in the affinity chromatography is composed of a matrix, a hydrophilic cross-linker and a ligand (R). In the structure, the matrix is cross-linked agarose, preferably highly cross-linked high-flow agarose, and the ligand (R) is a Camelidae-derived single-domain antibody fragment that is a protein binding to factor VII. The resin may selectively bind to a FVII-derived protein since the protein specifically binding to FVII is linked to an agarose fragment via a covalent bond. Since the ligand also has a long cross-linker showing hydrophilicity, the resin has a structure in which the ligand readily binds to a target protein to be isolated. Examples of the resin may include ‘VIISelect’ commercially available as a custom-designed medium (GE Healthcare; UK), but the present invention is not limited thereto. An exemplary VIISelect has the following properties.
Particle size: 75 μm (d₅₀ᵥ)
   Ligand: recombinant protein produced in Saccharomyces cerevisiae
Ligand density: 5.7 mg per 1 mL of medium
Binding capacity: 8 mg per 1 mL of medium
Working temperature: 4° C. to 30° C.

The affinity chromatography using the VIISelect is typically known to elute factor VII with an elution buffer (pH 7.5) including 50 mM Tris, 1.5 M sodium chloride, and 50% propylene glycol. However, the fusion protein including the factor VII according to one exemplary embodiment of the present invention has a problem in that the use of the eluate cause a significant decrease in yield. Therefore, the present invention is technically characterized in that an aqueous sodium thiocyanate (2.5 M) buffer at pH 5.0 to pH 8.0, preferably pH 7.0 is used instead of the conventional known eluates to enhance the isolation and purification of the fusion protein including the factor VII.

According to one exemplary embodiment of the present invention using the affinity chromatography, a resin stationary phase is equilibrated with a buffer (pH 7.5) including 50 mM Tris and 150 mM sodium chloride, and a non-residual fragment is then removed with a washing buffer until the resin stationary phase reaches an equilibrium (which is constantly retained at the baseline). Thereafter, the FVII fusion protein may be isolated and purified using an aqueous sodium thiocyanate (2.5 M) buffer (pH 7.0).

The recovery capacity of the FVII fusion protein may be determined by measuring the light absorbance at a wavelength of 280 nm. The activity of the recovered protein may be determined by measuring the amount of light scattered when red light (660 nm) passes through a mixed solution of sample plasma and a reagent, which indicates, a degree of blood coagulation, using a blood coagulation analyzer. According to one exemplary embodiment of the present invention, the FVII fusion protein having a purity of 95% or more may be recovered in a yield of 90% or more using the above-described affinity chromatography.

According to another exemplary embodiment, the FVII fusion protein of the present invention may be isolated and purified by mixed-mode chromatography using a ceramic fluoroapatite gel (Ca₁₀(PO₄)₆F₂) as a stationary phase.

A column using the ceramic fluoroapatite gel as the stationary phase is a column used to isolate proteins having a range of low pI values based on charge interaction. The column has a structure similar to the commercially available ceramic hydroxyapatite column manufactured by Novo. The ceramic fluoroapatite has a structure in which a hydroxyl (OH⁻) group of the ceramic hydroxyapatite is replaced with fluorine (F⁻). the fluorine ions are known to increase the stability of the resin at an acidic condition, compared to the hydroxyl groups.

The stationary phase of ceramic fluoroapatite is equilibrated with a buffer (pH 6.5) including 25 mM imidazole and 0.02% Tween 80. A non-residual fragment is removed with a washing buffer until the stationary phase reaches an equilibrium (which is constantly retained at the baseline). As the elution buffer to elute or for elution of the factor FVII, an aqueous buffer (pH 5.0 to 8.0) including sodium phosphate and sodium chloride, preferably an elution buffer (pH 6.3) including 25 mM imidazole, 0.02% Tween 80, and 180 to 340 mM Na-Pi may be used. The recovery capacity of the FVII fusion protein may be determined by measuring the light absorbance at a wavelength of 280 nm. The activity of the recovered protein may be determined by analyzing the chromogenic activity using COASET FVII (Chromogenix, #821900-63) analytic kit. In the present invention, the FVII fusion protein having a purity of 95% or more may be recovered in a yield of 90% or more by performing the anion exchange chromatography.

In the present invention, the fusion protein undergoing the affinity chromatography or the mixed-mode chromatography may be further isolated and purified with anion exchange chromatography using Q-sepharose FF gel as a stationary phase.

The Q-sepharose FF gel is a potent anion exchanger. In this case, when a net charge has a negative value at a certain pH, an amine group of a protein may bind to an amine exchanger through a charge exchange to remove a low level of non-specifically binding impurities of the host cells.

The stationary phase of the Q-sepharose FF gel is equilibrated with a buffer (pH 8.0) including 20 mM Tris, 20 mM sodium chloride, and 5 mM calcium chloride. A non-residual fragment is removed with a washing buffer until the stationary phase reaches an equilibrium (which is constantly retained at the baseline). As the elution buffer for elution of the activated factor FVII fusion protein, an aqueous buffer (pH 5.0 to 8.0) including 10 mM to 50 mM Tris, 1 mM to 10 mM calcium chloride, and 50 mM to 150 mM sodium chloride, preferably an aqueous buffer (pH 5.0 to 8.0) including 20 to 40 mM Tris, 2 to 5 mM calcium chloride, and 60 to 100 mM sodium chloride, and more preferably a buffer (pH 8.0) including 20 mM Tris, 5 mM calcium chloride, and 86.64 mM sodium chloride may be used.

The recovery capacity of the FVII fusion protein may be determined by measuring the light absorbance at a wavelength of 280 nm. The activity of the recovered protein may be determined by analyzing the chromogenic activity using COASET FVII (Chromogenix, #821900-63) analytic kit. The FVII fusion protein having a purity of 95% or more may be recovered in a yield of 50% or more by performing the anion exchange chromatography.

According to one exemplary embodiment of the present invention, the FVII fusion protein having a purity of 95% or more may be recovered from a cell culture medium in a yield of 50% or more using affinity chromatography using VIISelect or mixed-mode chromatography using ceramic fluoroapatite and anion exchange chromatography using a Q-sepharose FF gel.

### [Mode for Invention]

Hereinafter, the present invention will be described in further detail with reference to the following exemplary embodiments. However, it should be understood that the description proposed herein is just a preferable example for the purpose of illustrations only, not intended to limit the scope of the invention.

### EXAMPLE 1

### Construction of Factor VII (FVII) Plasmid Vector (pcDNA3.1-Hygro-FVII)

RNA purified from Hep G2 cells (KCLB No. 88065) was used as a template for reverse transcription. A cDNA transcript was amplified by PCR using FVII gene specific primers, FVII-F and FVII-R (SEQ ID NOs: 25 and 26) to obtain an open reading frame (ORF) of a human FVII gene. The PCR was as follows: 50 μL of a reaction solution (including 0.5 μL of cDNA, 0.4 μM of respective primers (10 pmol/μL) set forth in SEQ ID NOs: 25 and 26, 0.2 mM dNTP, 5 units of Taq DNA polymerase, and water) was reacted at 94° C. for 5 minutes; reacted for 35 cycles, with one cycle consisting of denaturation at 94° C. for 1 minute, annealing at 60° C. for 1 minute, and extension at 72° C. for 2.5 minutes; and then reacted at 72° C. for 5 minutes to terminate the reaction. The purified PCR product was cloned into a pGEM-T easy vector (Promega, Cat No.: A1360). The cloned vector was treated with restriction endonucleases EcoRI and NcoI to screen positive clones. The screened clones were verified by DNA sequencing. To transfer an ORF of the FVII (FVII-ORF) to an expression vector, FVII-ORF of a pGEM-T easy vector was digested with a restriction endonuclease NotI. The FVII-ORF digested with NotI was treated with T4 DNA polymerase to make blunt ends and ligated to a pcDNA3.1-hygro vector (Invitrogen) treated with restriction endonucleases HindIII/XbaI. The ligated of vector and FVII-ORF fragment were verified by treatment with restriction endonucleases ApaI, XbaI, EcoRI, NcoI, and PstI and by DNA sequencing. This vector was named “pcDNA3.1-hygro-FVII.”

### EXAMPLE 2

### Construction of FVII-Tf Expression Vector (pcDNA3.1-Hygro-FVII-Tf)

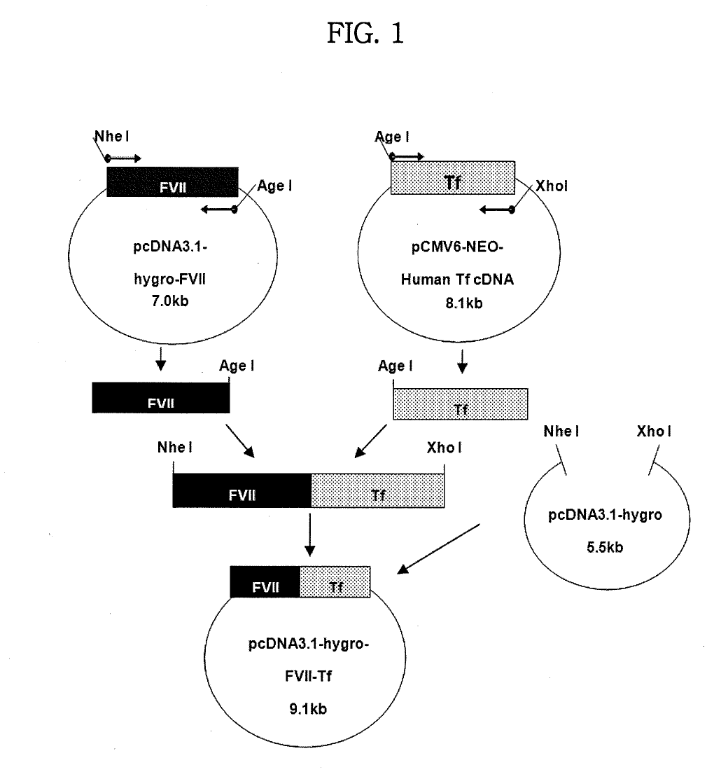

The FVII cDNA prepared in Example 1 was connented to human transferrin (Tf) cDNA and expressed as a single zymogen in animal cells. Human transferrin cDNA was purchased from Origene (Cat No.: SC322130) to obtain cDNA having the same sequence as GenBank Accession No: NM_{—}001063.2. Primers used for ligation were designed to remove a stop codon of FVII and nucleotides coding for a signal peptide of Tf. Thereafter, to insert linkers having various sizes between FVII and Tf, an AgeI site (ACCGGT) translated into threonine (Thr) and glycine (Gly) was added to a linking primer. A fusion protein has a structure of (leader peptide)-(mature FVII)-(Thr-Gly)-(mature Tf) (the leader peptide includes a combination of a signal peptide (a prepeptide) not present in mature FVII and a peptide (a propeptide) cleaved by a processing enzyme, consists of 38 amino acids and corresponds to the amino acids at the 1^{st} to 38^{th} positions in the amino acid sequence set forth in SEQ ID NO: 1). The cDNAs of FVII and Tf were amplified using the primers FVII-S1, FVII-AS1, Tf-S1, and Tf-AS1 (SEQ ID NOs: 27 to 30), and the vectors described in Example 1 were used as the vector. The primers set forth in SEQ ID NOs: 27 and 30 include NheI and XhoI sites, respectively. A cloning scheme in which FVII cDNA is ligated to Tf cDNA is shown in FIG. 1. First, FVII cDNA was amplified from the pcDNA3.1-hygro-FVII vector prepared in Example 1 using PCR. The PCR was as follows: 50 μL of a reaction solution (including 1 μL of a mixed vector template, 1 μL of primers FVII-S1 and FVII-AS1 (10 μM each), 10 μL of a 5× Phusion HF buffer, 200 μM dNTP, 0.5 μL of a Phusion DNA polymerase (FINNZYMES, #F-530S, 2 units/μL), and 35.5 μL of water) was reacted at 98° C. for 30 seconds; reacted for 30 cycles, with one cycle consisting of denaturation at 98° C. for 10 seconds, annealing at 60° C. for 45 seconds, and extension at 72° C. for 30 seconds; and then reacted at 72° C. for 7 minute to terminate the reaction.

Next, Tf was amplified using human transferrin cDNA as a template. The reaction was performed in the same manner as in the FVII PCR conditions, except that the primers Tf-S1 (10 μM) and Tf-AS1 (10 μM) were used.

The amplified FVII and Tf cDNAs were linked through a series of restriction endonuclease digestions and ligations. Each of the cDNA fragments amplified by PCR was treated with the restriction endonucleases AgeI and XhoI, or NheI. The cDNA fragments of the FVII and Tf treated with the restriction endonuclease(s) was purified and ligated at a molar ratio of 1:1. The ligated DNA was sub-cloned into pcDNA3.1-hygro vector (Invitrogen) treated with the restriction endonucleases NheI/ShoI. The size and sequence of the insert fragment were verified by DNA sequencing.

### EXAMPLE 3

### Construction of FVII-GS Linker-Tf Expression Vector

A peptide consisting of 5 amino acids including glycine and serine was used as a basic linker unit. The basic linker unit includes four glycine residues and one serine residue such as a ‘GGGGS.’ The basic GS linker unit (hereinafter referred to as “GS-X linker” where X represents the number of repeats of the basic GS linker unit) was used to construct longer GS linkers. In the present invention, linkers ranging from GS-1 to GS-15 were constructed.

1) Construction of FVII-GS-1 Linker-Tf Expression Vector

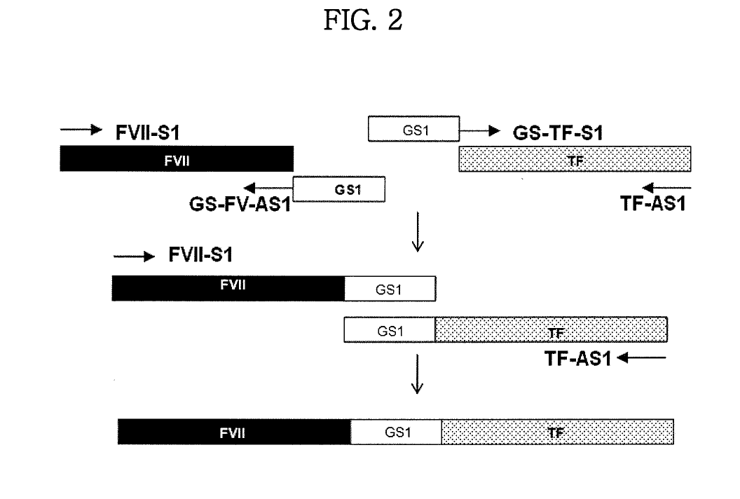

Primers GS-FV-AS1 and GS-Tf-S1 (SEQ ID NOs: 31 and 32) including a sequence of the basic GS linker unit were synthesized, and a GS-1 linker was inserted between FVII and Tf by overlapping PCR (see FIG. 2).

PCR was performed with Phusion DNA polymerase (FINNZYMES, #F-530S) using the primers FVII-S1 and GS-FV-AS1 (SEQ ID NOs: 27 and 31) to ligate the GS-1 linker to FVII. The PCR was as follows: 50 μL of a reaction solution (including 1 μL of a pcDNA3.1-hygro-FVII-Tf vector, 1 μL of FVII-S1 (10 pmole/μL), 1 μL of GS-FV-AS1 (10 pmole/μL), 1 μL of 10 mM dNTP, 10 μL of a 5× Phusion HF buffer, 35.5 μL of water, 0.5 μL of Phusion DNA polymerase (2 unit/μL)) was reacted at 98° C. for 30 seconds; reacted for 30 cycles, with one cycle consisting of denaturation at 98° C. for 10 seconds, annealing at 64° C. for 30 seconds, and extension at 72° C. for 45 seconds; and then reacted at 72° C. for 7 minute to terminate the reaction. Meanwhile, the PCR was performed in the same manner, except that the primers (SEQ ID NO: 32 and 30) for GS-Tf-S1 and Tf-AS1 were used instead of the primers in the reaction solution to ligate Tf to the GS-1 linker. Overlapping PCR was performed using the amplified PCR products as templates. The overlapping PCR was as follows: a reaction solution including 1 μL of each of the amplified PCR products, 1 μL of FVII-S1 (10 pmole/μL, SEQ ID NO: 27), 1 μL of an antisense primer (Tf-AS1 10 pmole/μL, SEQ ID NO: 30), 10 μL of a 5× Phusion HF buffer, 1 μL of 10 mM dNTP, 34.5 μL of water, and 0.5 μL of Phusion DNA polymerase (2 units/μL) was reacted at 98° C. for 1 minute; reacted for 45 cycles, with one cycle consisting of denaturation at 98° C. for 10 seconds, annealing at 66/68° C. for 30 seconds, and extension at 72° C. for 45 seconds; and then reacted at 72° C. for 7 minute to terminate the reaction. The amplified overlapping PCR product was digested with the restriction endonucleases NheI and Xhol and cloned into a pcDNA3.1-hygro-lacZ vector.

2) Construction of FVII-GS-3 Linker-Tf Expression Vector

Primers GS3-S and GS3-AS (SEQ ID NOs: 33 and 34) including GS-3 and AgeI sites were synthesized. To prepare a GS-3 double helix linker, 5 μL of GS3-S (100 pmole/μL), 5 μL of GS3-AS (100 pmole/μL), 2 μL of a 10× annealing buffer (100 mM Tris-Cl [pH 8.0], 1 M NaCl, and 10 mM EDTA), and 8 μL of water were mixed, heated at 98° C. for 10 minutes, and then cooled at 25° C. for 1 hour to perform annealing. The annealed linker was digested with the restriction endonuclease AgeI, and the pcDNA3.1-hygro-FVII-Tf vector prepared in Example 2 was also digested with AgeI. The digested vector was treated with 1 μL of calf intestinal phosphatase (CIP; NEB, #M0290S) at 37° C. for 1 hour, subjected to a gel extraction procedure (QIAGEN, #28704), and ligated with the annealed linker at a molar ratio of 1:3 (vector:insert) using a T4 DNA ligase (TAKARA, #2011A).

3) Construction of FVII-GS-5 Linker-Tf Expression Vector and FVII-GS-15 Linker-Tf Expression Vector

A new method was introduced to construct a fusion protein expression vector including a GS-5 linker. A FVII-Tf fusion vector including a linker extended through the following two steps was constructed.

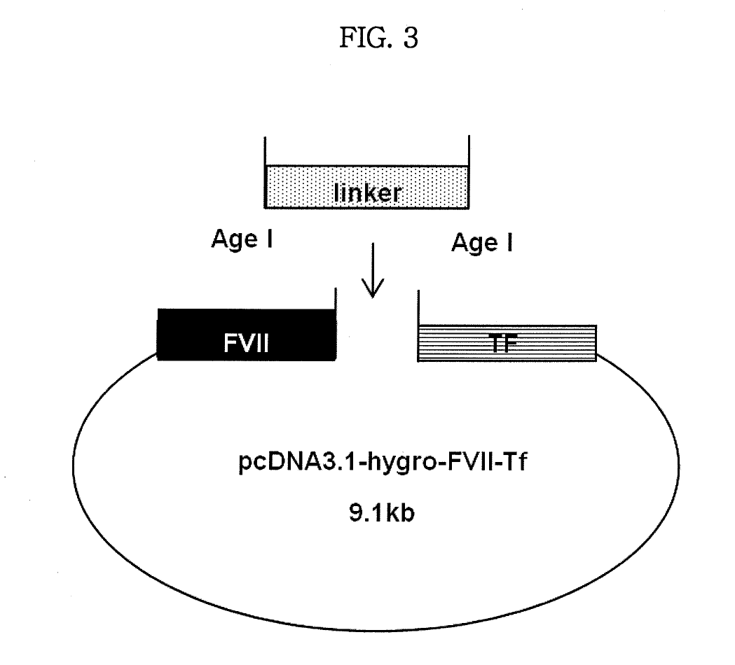

The first step is to add a synthesized double-stranded (ds) GS-2 linker to the GS-3 linker obtained above. After the extension of the linker was confirmed, the linker was digested and inserted between FVII and Tf genes of the pcDNA3.1-hygro-FVII-Tf vector. To extend the GS-3 linker to a GS-5 linker, for example, a synthesized dsGS-2 unit set forth in SEQ ID NO: 35 was treated with BglII and then ligated to a pcDNA3.1-hygro-FVII-GS-3-Tf vector treated with the restriction endonucleases BamHI and StuI. Subsequently, the extension of the linker was verified through the treatment with BamHI and AgeI, and the extended linker was then digested with AgeI, and sub-cloned into a pcDNA3.1-hygro-FVII-Tf vector treated with AgeI and CIP. FVII-Tf fusion expression vectors including the GS-7, GS-9, GS-11, GS-13, and GS-15 linkers were also constructed in the same manner (see FIG. 3).

### EXAMPLE 4

### Construction of FVII-Tf Expression Vector (pcDNA3.1-Hygro-FVII-GS1-T-Tf) Including Linker Containing Thrombin Restriction Site

Thrombin cleavage sites were linked to both ends of the GS-1 unit (hereinafter referred to as a “GS1-T linker”). A dsGS1-T linker set forth in SEQ ID NO: 36 (sense) was synthesized so that both ends of the dsGS1-T linker had AgeI sites. The dsGS1-T linker was treated with AgeI and purified using a PCR purification kit (Qiagen, Cat. No.: 28104). The purified linker was ligated to pcDNA3.1-hygro-FVII-Tf vector treated with CIP/AgeI.

### EXAMPLE 5

### Construction of FVII-Tf Expression Vector (pcDNA3.1-Hygro-FVII-Helix-Tf) Including Helix Linker

A helix linker DNA was prepared using the method disclosed in US Patent Application No: 2009/0170163. AgeI sites were added to both ends of the prepared helix linker DNA using the primers Helix linker S and Helix linker AS (SEQ ID NOs: 37 and 38). A helix linker obtained by annealing the primers Helix linker S and Helix linker AS (SEQ ID NOs: 37 and 38) was treated with AgeI and then inserted into a pcDNA3.1-hygro-FVII-Tf vector treated with AgeI and CIP. The prepared pcDNA-hygro-FVII-Helix-Tf vector was verified by DNA sequencing.

The characteristics of the expression vectors constructed in Examples 2 to 5 are listed in the following Table 1.

TABLE 1

| | | | | Number |
|---|---|---|---|---|
| | C- | | N-terminus | of amino |
| FVII fusion | terminus | Linker sequence (sequence | of fusion | acids in |
| protein | of FVII | identification number) | partner | linker |
| FVII-Tf | APFP | — | VPDKTV | 0 |
| FVII-GS1-Tf | APFP | GGGGS (SEQ ID NO: 3) | VPDKTV | 5 |
| FVII-GS3-Tf | APFP | (GGGGS)₃ (SEQ ID NO: 4) | VPDKTV | 15 |
| FVII-GS5-Tf | APFP | (GGGGS)₅ (SEQ ID NO: 5) | VPDKTV | 25 |
| FVII-GS7-Tf | APFP | (GGGGS)₇ (SEQ ID NO: 6) | VPDKTV | 35 |
| FVII-GS9-Tf | APFP | (GGGGS)₉ (SEQ ID NO: 7) | VPDKTV | 45 |
| FVII-GS11-Tf | APFP | (GGGGS)₁₁ (SEQ ID NO: 8) | VPDKTV | 55 |
| FVII-GS13-Tf | APFP | (GGGGS)₁₃ (SEQ ID NO: 9) | VPDKTV | 65 |
| FVII-GS15-Tf | APFP | (GGGGS)₁₅ (SEQ ID NO: 10) | VPDKTV | 75 |
| FVII-GS1-T-Tf | APFP | GGGGSLVPRGSGGGS (SEQ | VPDKTV | 15 |
| | | ID NO: 12) | | |
| FVII-Helix-Tf | APFP | GA(EAAAK)₄A (SEQ ID NO: | VPDKTV | 23 |
| | | 11) | | |

* Thr-Gly derived from AgeI is present in function protein FVII-Tf.
* Thr derived from AgeI is present upstream of the linker sequences set forth in SEQ ID NOs: 4 to 12, and Thr-Gly derived from AgeI is present downstream of the linker sequences.

### EXPERIMENTAL EXAMPLE 1

### Measurement of Specific Activity of FVII-Fusion Protein

The FVII-fusion proteins prepared in Examples 2 to 5 were expressed in CHO (VK2) cells that are derived from the CHO cell line in which vitamin K epoxide reductase complex subunit 1 (VKORC1) is stably expressed.

The expression vectors constructed in Examples 2 to 5 were purified using Endo-free plasmid maxi kit (Qiagen, #27104). β-Galactosidase was used as the control for transfection. The CHO (VK2) cells were seeded in a 6-well plate at a concentration of 1.5×10⁶/well. The cells were cultured for 24 hours in α-MEM (Lonza, #12-169F) supplemented with 10% FBS (Lonza, #14-501F), 1× HT (Invitrogen, #11067-030), 4 mM L-glutamine (Lonza, #17-605E), and 200 μg/mL of hygromycin (Invitrogen, #10687-010), and the cells were transfected with Lipofectamine 2000 (Invitrogen) according to the supplier's manual. After 4 hours of transfection, the culture medium was replaced with a serum-free culture medium (OptiMEM), and 5 μg/mL of vitamin K was added thereto. After 48 hours of the culture, the culture medium was sampled and stored at −70° C.

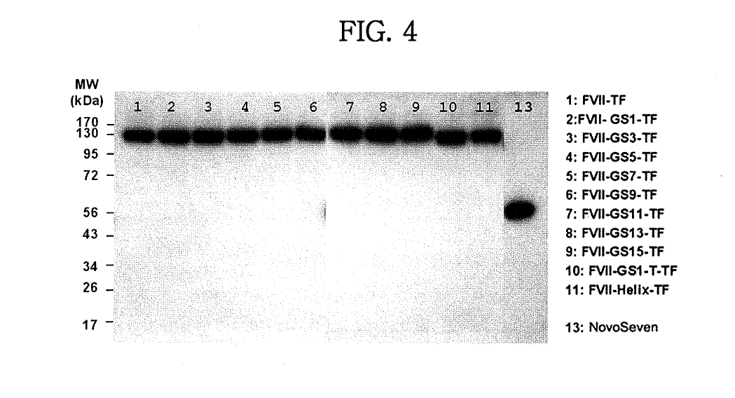

The chromogenic activities of the expressed FVII-fusion proteins and the amounts of the antigens were analyzed using COATEST Factor VII analytic kit (Chrmogenix, #821900-63) and a FVII ELISA kit (Cedarlene Lab, #CL20030K), respectively. The analysis was performed according to the manufacturer's manual. The standard human plasma standardized for the WHO international standard was used as the control FVII for quantitative analysis. The expression of proteins was confirmed by Western blotting technique using FVII and Tf antibodies. Based on the ELISA results, that the expressed FVII-fusion proteins had an expected size without any fragmentation when the same amount of the FVII fusion proteins was loaded (see FIG. 4).

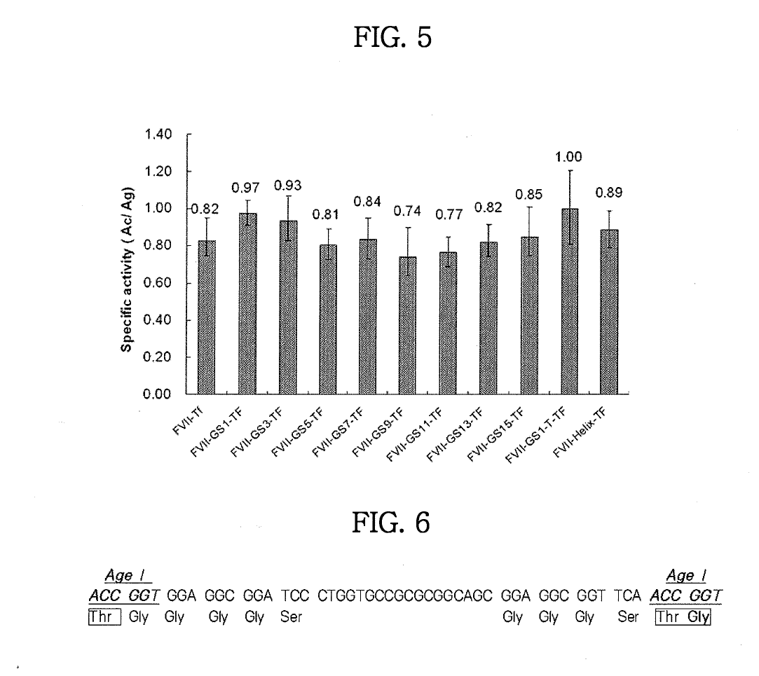

Meanwhile, the specific activities of the FVII-transferrin fusion proteins were in a range of 0.74 to 1 (see Table 2). The FVII-transferrin fusion proteins including the linker also had FVII activity of 70% or more. The length of the linker and the specific activities of the FVII fusion proteins were shown to have no certain relationship, but the FVII fusion proteins including a shorter linker had a slightly higher specific activity than the FVII fusion proteins including a longer linker. Especially, the FVII-GS1-Tf and FVII-GS1-T-Tf fusion proteins had a remarkably high specific activity (see FIG. 5).

TABLE 2

| FVII fusion | | | Specific activity |
|---|---|---|---|
| protein | Antigen (%) | Activity (%) | (activity/antigen) |
| FVII-Tf | 53.2 ± 5.0 | 43.9 ± 0.3 | 0.82 |
| FVII-GS1-Tf | 53.4 ± 3.1 | 52.0 ± 0.5 | 0.97 |
| FVII-GS3-Tf | 61.9 ± 8.0 | 57.7 ± 0.2 | 0.93 |
| FVII-GS5-Tf | 69.3 ± 5.6 | 55.9 ± 1.4 | 0.81 |
| FVII-GS7-Tf | 70.9 ± 8.2 | 59.3 ± 1.1 | 0.84 |
| FVII-GS9-Tf | 64.2 ± 8.6 | 47.5 ± 0.7 | 0.74 |
| FVII-GS11-Tf | 59.1 ± 3.9 | 45.3 ± 0.9 | 0.77 |
| FVII-GS13-Tf | 59.7 ± 5.1 | 49.1 ± 0.8 | 0.82 |
| FVII-GS15-Tf | 59.2 ± 6.0 | 50.2 ± 0.5 | 0.85 |
| FVII-GS1-T-Tf | 70.8 ± 8.7 | 71.0 ± 2.6 | 1.00 |
| FVII-Helix-Tf | 89.0 ± 5.7 | 78.9 ± 2.2 | 0.89 |

### EXAMPLE 6

### Characterization of FVII Fusion Protein According to Tf Fusion Direction

To observe the differences in characteristics of the fusion proteins according to the fusion direction, a fusion protein in which human transferrin (Tf) was linked to the N-terminus of FVII was prepared, and its activity was compared to a fusion protein in which human transferrin (Tf) was linked to the C-terminus of FVII. Specific procedures were as follows.

<6-1> Construction of Tf-FVII and Tf-GS1T-FVII Expression Vectors

Two fusion proteins in which Tf was linked to the N-terminus of FVII were prepared. In this case, the fusion proteins had the following structures: 1) (leader peptide of Tf)-(mature Tf)-(Thr-Gly)-(mature FVII); and 2) (leader peptide of Tf)-(mature Tf)-(Thr)-(GS1-T; SEQ ID NO: 12)-(Thr-Gly)-(mature FVII).

To obtain a gene sequence of Tf including a coding n.t sequence for the leader peptide, first, a forward primer (Nhe-Tf: SEQ ID NO: 46) used for amplification was designed to include a NheI site for the purpose of cloning, and a reverse primer (Tf-Age: SEQ ID NO: 47) was designed to remove a stop codon of Tf and to include a NheI site for the purpose of cloning as well. A forward primer (Age-FVII: SEQ ID NO: 48) used to clone mature FVII from which the leader peptide was removed was designed to include a restriction endonuclease AgeI, and a reverse primer (VII-Xho: SEQ ID NO: 49) was designed to include a restriction endonuclease XhoI.

In the case of the Tf gene, cDNA purchased from Origene (Cat No.: SC322130) was used as a PCR template as described in Example 2. The PCR was performed as follows: 50 μL of a reaction solution (including 1 μL of vector template, 2 μL of primers Nhe-Tf and Tf-AgeI (10 μM for each), 10 μL of a 5× Phusion HF buffer, 1 μL of 10 mM dNTP, 0.5 μL of Phusion DNA polymerase (FINNZYMES, #F-530S, 2 units/μL), and 33.5 μL of water) was reacted at 98° C. for 30 seconds; reacted for 25 cycles, with one cycle consisting of denaturation at 98° C. for 10 seconds, annealing at 70° C. for 30 seconds, and extension at 72° C. for 36 seconds; and then reacted at 72° C. for 10 minute to terminate the reaction. FVII was amplified by PCR using the pcDNA3.1-hygro-FVII-GS1-T-Tf vector constructed in Example 4 as the template. The reaction was performed in the same manner as in the Tf PCR conditions, except that the primers Age-FVII (10 μM) and VII-Xho (10 μM) were used.

The Tf gene amplified by the PCR was digested with the NheI/AgeI restriction endonucleases, and the digested fragment of the Tf PCR product was inserted into a pcDNA3.1-hygro-FVII-GS1T-Tf vector to obtain a pcDNA3.1-hygro-Tf-Tf vector. The obtained pcDNA3.1-hygro-Tf-Tf vector and the FVII PCR products were treated with the AgeII Xhol restriction endonucleases, and then the vector and the FVII PCR product were ligated to construct an expression vector including the pcDNA3.1-hygro-Tf-FVII fusion protein. The pcDNA3.1-hygro-Tf-GS1-T-FVII expression vector was constructed by treating the dsGS1-T sequence synthesized as in Example 4 with the AgeI restriction endonuclease and by inserting the dsGS1-T sequence into the obtained pcDNA3.1-hygro-Tf-FVII. The restriction endonuclease mapping and nucleotide sequencing confirmed that the obtained expression vectors were constructed as intended.

<6-2> Expression and Characterization of Fusion Protein

To characterize the proteins in which Tf was fused to the C-terminus of FVII and the proteins in which Tf was fused to the N-terminus of FVII, each of the expression vectors (pcDNA3.1-hygro-FVII-Tf, pcDNA3.1-hygro-FVII-GS1T-FVII, pcDNA3.1-hygro-Tf-FVII, and pcDNA3.1-hygro-Tf-GS1-T-FVII) was temporarily expressed in CHO cells.

The 4 constructed plasmid DNAs were isolated using Endo-free maxi prep kit (Qiagen). Before a day of transfection, the CHO (DG44) cells cultured in a T75 flask were treated with trypsin to be detached from the T75 flask, and then seeded in a 6-well plate at a concentration of 1.5 ×10⁶ cell/well. After 24 hours, the cells were transfected according to the manufacturer's manual. After 4 hours of transfection, the medium was removed from each well, and replaced with 2 mL of a growth medium supplemented with 5 μg/mL of vitamin K. After the transfection, the cells in the 6-well plate were cultured at 37° C. in a 5% CO₂ incubator. After 48 hours, the culture medium was centrifuged to obtain supernatant. Then, the supernatant was divided into 1.5 mL tubes, stored at −70° C., and used for a FVII chromogenic assay and FVII ELISA. The medium-free plate was washed with 2 mL of HBSS per a well, and 250 μL of a lysis solution (Tropix, #ABX210LM, supplemented with 1 mM DTT) was evenly spread on the plate and then stored at −70° C. for β-galactosidase analysis.

The FVII chromogenic assay and FVII ELISA were performed in the same manner as in Experimental Example 1. A medium stored in a freezer after the transfection was thawed just before the experiment and centrifuged to obtain a supernatant. Then, the supernatant was used as a sample to be analyzed. Standard human plasma (Dade Behring, #ORKL13, Lot #503216F) was used as the standard product for the analysi.

The measurement results are listed in the following Table 3. In the case of the Tf-FVII and Tf-GS1T-Tf fusion proteins in which Tf was linked to the N-terminus of FVII, no activity was measured, unlike the FVII-Tf and FVII-GS1T-Tf fusion proteins in which Tf was linked to the C-terminus of FVII. Also, when the amount of the fusion proteins was measured by FVII ELISA, the amount of the fusion proteins in which Tf was linked to the N-terminus of FVII was detected at a low level. However, when the fusion proteins were subjected to Western blotting using polyclonal antibodies against Tf, the fusion proteins were detected with similar detection sensitivity, regardless of the fusion direction. The results showed that, when Tf is fused to the N-terminus of FVII, the fusion proteins was normally translated, but the fusion protein had no FVII activity.

TABLE 3

| | FVII | FVII | Specific activity |
|---|---|---|---|
| Fusion proteins | activity (%) | antigen (%) | (activity/antigen) |
| FVII-Tf | 33.8 ± 0.73 | 37.0 ± 2.17 | 0.92 |
| Tf-FVII | Activity not | 8.0 ± 1.51 | — |
| | measurable | | |
| FVII-GS-1-T-Tf | 46.6 ± 0.29 | 43.0 ± 4.75 | 1.08 |
| Tf-GS-1-T-Tf | Activity not | 13.5 ± 1.01 | — |
| | measurable | | |

### EXAMPLE 7

### Characterization of Fusion Protein According to the Removal of Restriction Endonuclease Recognition Sequence Used for Fusion

To promote the insertion of various linkers between FVII and Tf upon fusion of FVII and Tf in Example 2, a restriction endonuclease (AgeI) recognition sequence was used. As a result, some fusion proteins had Thr and Gly residues encoded by the restriction endonuclease at both ends of the linker. Accordingly, in this example, it was checked whether the properties of the fusion proteins were changed according to the presence of the restriction endonuclease (AgeI) recognition sequence.

In this example, the FVII-GS1-T-Tf fusion protein including the GS1-T linker were subjected to PCR-based site-directed mutagenesis, using a mutagenic primer, to remove the restriction endonuclease recognition sequence. As shown in FIG. 6, the Thr residue upstream of the linker and the Thr-Gly residues downstream of the linker were removed. In this case, the primers used are listed in the following Table 4.

TABLE 4

| | | SEQ |
|---|---|---|
| Kinds of | | ID |
| primers | Sequences (5′->3′) | NOs |
| TG del-S | CAG CGG AGG CGG TTC AGT CCC TGA TAA | 50 |
| | AAC TG | |
| TG del-AS | CAG TTT TAT CAG GGA CTG AAC CGC CTC | 51 |
| | CGC TG | |
| T del-S | CGA GCC CCA TTT CCC GGT GGA GGC GGA | 52 |
| | TC | |
| T del-AS | GAT CCG CCT CCA CCG GGA AAT GGG GCT | 53 |
| | CG | |

<7-1> Thr-Gly Removal

Mutagenesis was performed using a PCR method. The PCR conditions was as follows: 1 μL of pcDNA3.1-hygro-FVII-GS1T-Tf vector, 0.2 μL of a sense primer (TG del-S 10 μM), 0.2 μL of an antisense primer (TG del-AS 10 μM), 1 μL of 10 mM dNTP, 4 μL of a 5× PCR buffer, 14 μL of distilled water, and 0.2 μL of Phusion DNA polymerase (FINNZYMES, #F-530S) were added and reacted at 98° C. for 30 seconds (one cycle); reacted for 18 cycles, with one cycle consisting of denaturation at 98° C. for 10 seconds, annealing at 58° C. for 30 seconds, and extension at 72° C. for 3 minutes; and then reacted at 72° C. for 7 minute (one cycle). To remove the original template DNA, 1 μL of DpnI (NEB, #R0176S) was added to the previously amplified PCR products, and the resulting reaction solution was then incuvated at 37° C. for 1 hour. 50 μL of HIT competent cells (DH5α, RH617) was transformed with 10 μL of the DNA treated with DpnI and cultured overnight in an LB+amp (10 mg/mL) solid medium. The N.T sequences of the 4 clones obtained from the transformation were analyzed. Among them, the two clones were confirmed to be mutated.

<7-2> Thr Removal

Mutagenesis was performed in the same manner as in Example <7-1> except using different kinds of primers. That is, PCR-based mutagenesis was performed under the same condition using 1 μL of plasmid DNA in the clone whose mutation was confirmed in Example <7-1> as the template and using 1 μL of a sense primer (T del-S 10 pmole) and 1 μL of an antisense primer (T del-AS 10 pmole). The 4 selected clones were subjected to nucleotide sequencing. Among these, the 3 clones were confirmed to be mutated. The resulting expression vector was named “pcDNA3.1-hygro-FVII-GS1-T-Tf(M3).”

<7-3> Characterization of Fusion Protein from which Restriction Endonuclease Recognition Sequence is Removed

CHO cells were transfected with the FVII-GS1-T-Tf and the FVII-GS1-T-Tf(M3) expression vectors to obtain medium supernatants. The obtained supernatants were subjected to an FVII chromogenic assay (Chromogenix) and FVII ELISA (Cedarlane) to determine a change in activity/antigen ratio. As a result, the amount of the antigens and the activities of the FVII-GS1T-Tf and FVII-GS1T-Tf(M3) fusion proteins were almost identical, and there was no change in activity/antigen ratio (specific activity), as listed in Table 5.

TABLE 5

| | FVII | FVII | Specific activity |
|---|---|---|---|
| | antigen (%) | activity (%) | (activity/antigen) |
| FVII-GS1-T-Tf | 34.1 ± 2.1 | 39.6 ± 2.2 | 1.16 |
| FVII-GS1-T-Tf(M3) | 33.5 ± 4.7 | 38.0 ± 0.7 | 1.14 |

### EXAMPLE 8

### Measurement of Half-Life of the Fusion Protein

This example was performed to check an effect of improving the half-lives of the fusion proteins according to one exemplary embodiment of the present invention. The FVII-Tf, FVII-GS1-Tf, FVII-GS3-Tf, FVII-GS15-Tf, and FVII-GSI-T-Tf fusion proteins were used as the fusion proteins used in this example, and wild-type FVII and commercially available FVIIa (NovoSeven®, Novo Nordisk), were used as non-fusion protein as the coltrols.

<8-1> Sample Preparation

1) Preparation of Expression Medium

Five expression vectors for FVII fusion proteins in which FVII and Tf were fused with each other and an expression vector for FVII which is not a fusion protein were expressed in a FreeStyle CHO-S cell line (Invitrogen, Cat. No.: R800-07). The CHO-S cells were suspended and cultured in a spinner flask containing a FreeStyle CHO expression medium supplemented with 8 mM L-Glu (GIBCO, L-glutamine 200 mM (100×), Cat. No.: 25030-081). The cultured cells were seeded at a density of 4×10⁵ cells/mL before 24 hours of transformation, and the cells were transfected when the density of the cells had reached 1×10⁶ cells/mL. The DNA used for transfection was prepared using Endo-free maxi prep kit (QIAGEN, Cat No.: 12362) or Endo-free plasmid prep kit (QIAGEN, Cat No.:12381), and the transfection was performed according to the transfection protocol with FreeStyle MAX reagent (Invitrogen, Cat No.: 16447-100) transfection protocol. 500 μg of DNA was added to 8 mL of OptiPRO SFM (Invitrogen, Cat No.: 12309-019) and then mixed. Also, 8 mL of OptiPRO SFM (Invitrogen, Cat No.: 12309-019) and 500 μL of a FreeStyle MAX reagent were added into another tube, and OptPRO SFM and FreeStyle MAX reagent were carefully mixed, and kept at room temperature for 10 minutes. After 10 minutes, FreeStyle CHO-S cells were transfected with each of the mixtures. The transfected cells were cultured at 37° C. for 3 to 5 days in a 5% CO₂ incubator, and the supernatants were fractionated.

2) Purification of the Expression Medium

To remove the cells and cell debris remaining in the culture medium obtained by the spinner flask culture, the culture medium was filtered through a 0.22 μm filter (Corning). The filtered culture medium was 10 times concentrated by ultrafiltration using a tangential-flow membrane (Satorious, 30 kDa). The concentrated medium was applied to an XK16/20 (GE healthcare) column packed with ceramic hydroxyapatite (BIO-RAD, 157-0040) resin. The hydroxyapatite column was equilibrated with 10× column volume of an equilibration buffer (25 mM imidazole, 0.02% Tween 80, and 150 mM NaCl, pH 6.5) before the application of the concentrated medium. The concentrated medium was allowed to flow through the column, and the equilibration buffer, first washing buffer (25 mM imidazole, 0.02% Tween 80, and 100 mM sodium phosphate, pH 6.3), and second washing buffer (25 mM imidazole, 0.02% Tween 80, 100 mM sodium phosphate, and 1 M NaCl, pH 6.3) were then allowed to flow through the column to wash off the impurities. When the washing was completed, the fusion protein bound to the column was eluted with an elution buffer (25 mM imidazole, 0.02% Tween 80, and 500 mM sodium phosphate, pH 6.3). The eluate was replaced with Q-equilibration buffer (25 mM Histidine, 0.02% Tween 80, and 25 mM NaCl, pH 6.0) using a diafiltration method, and the elute was applied to a previously packed Hitrap Q HP (1.6×2.5 cm, 5 mL) column. Thereafter, the column was washed with Q-equilibration buffer, and the FVII/fusion protein bound to the column was then eluted with Q-elution buffer (25 mM histidine, 0.02% Tween 80, 0.025 mM CaCl₂, and 1 M NaCl, pH 6.0). The eluted protein was analyzed by a FVII-chromogenic assay, FVII ELISA, and SDS-PAGE/Western blotting method.

<8-2> Western Blotting Assay

The FVII and FVII/Tf fusion proteins partially purified through the 2-step column were confirmed to have a purity of 45% or more, as measured by SDS-PAGE/Coomassie blue staining. Since the human FVII remaining in an animal test was analyzed using an ELISA method, the presence of FVII-derived fragments in the purified fusion protein was confirmed by Western blotting. NovoSeven® (Novo Nordisk, 1.2 mg/vial, 60 KIU) and the purified sample were prepared at 0.1 IU (FVII activity)/10 μL, and the resulting sample solution was subjected to SDS-PAGE using a NuPage 4-12% bis-Tri gel (Invitrogen). After the electrophoresis was completed, the proteins separates by the SDS-PAGE was transferred to a PVDF membrane, and the membrane was blocked at room temperature for 1 hour in 10 mL with blocking buffer (25 mM Tris, 150 mM NaCl (pH 7.2), 5% skin milk, and 0.1% Tween 80). The blocking solution was discarded, and 10 mL (5% skim milk in PBS-T) of anti-FVII antibodies (Cat. No.: F8146, Sigma) or mouse anti-transferrin antibodies (sc52256, Santa Cruz) was added to the membrane at a ratio of 1:5000 and 1:500, respectively, and the containing the membrane and antibodies solution was stirred for 1 hour. the membrane was washed four times with a washing solution (25 mM Tris, and 150 mM NaCl, pH 7.2) and then stirred for 1 hour in 10 mL (5% skim milk in PBS-T) in a solution supplemented with secondary antibodies, goat anti-mouse IgG-HRP antibody (Cat. No 021040, Invitrogen), at a ratio of 1:50,000. The membrane was washed four times with a washing solution (25 mM Tris, and 150 mM NaCl, pH 7.2), and 2 mL of Super-signal west Femto mix (Thermo) was added to the membrane and reacted for 5 minutes. After the reaction was completed, the film was developed.

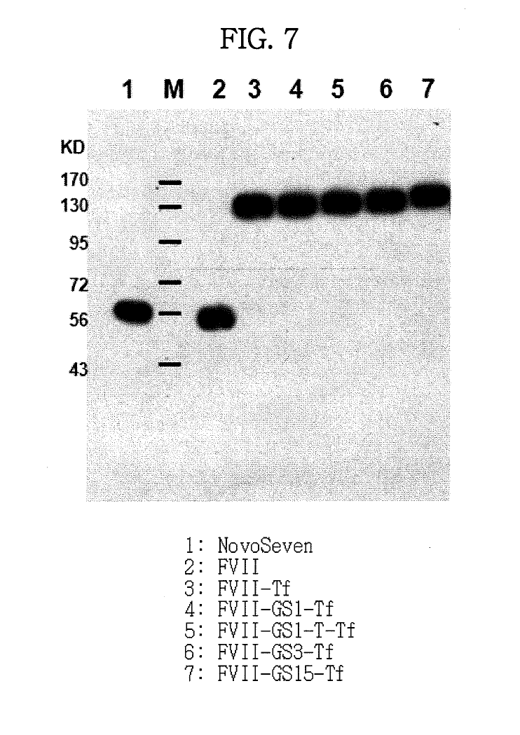

The Western blotting results are shown in FIG. 7. As shown in FIG. 7, no FVII-derived fragments was present in the purified protein and no Tf-derived fragments was present in the transferrin blot.

<8-3> Half-Life Measurement

The half-lives of the five fusion proteins (for example, one fusion protein having no linker and four fusion proteins having the linkers (GS1, GS1-T, GS3, and GS15)) and the half-lives of the two controls (for example, wild-type FVII expressed and purified under the same conditions and commercially available NovoSeven®) were compared in rats. The analysis of samples to be administered and to be acquired were performed by human FVII ELISA (Cedarlane, Paired Antibodies for ELISA Factor VII, #CL20030K) according to the manufacturer's guideline. The concentration of the sample to be administered was determined as an average value of the values measured in triplicate after the sample was diluted with an administration/dilution solution (3 mg/mL of NaCl, 1.5 mg/mL of CaCl₂ dihydrate, 1.3 mg/mL of glycylglycine, 0.1 mg/mL of Polysorbate 80, and 30 mg/mL of mannitol, pH 5.5). After the FVII ELISA quantification, each of the fusion proteins was diluted with the administration/dilution solution and administered intravenously into the tails of rats at a dose of 150 IU/kg, based on the weights of the rats (Sprague-Dawley rats weighing 250 to 300 g) measured on the day of experiments (groups of three rats according to the type of FVII). Blood was drawn a total of 11 times before drug administration, 5 minutes, 15 minutes, 30 minutes, 60 minutes, 1.5 hours, 2 hours, 4 hours, 6 hours, 8 hours, and 24 hours after the drug administration. 225 μL of the drawn blood and 25 μL of 3.2% sodium citrate were mixed and centrifuged at 4° C., 13,000 rpm for 1 minute to obtain supernatant, which was then stored at −70° C. The sample was diluted at a ratio of 1/50 or 1/100 with a washing buffer used in FVII ELISA kit (Cedarlane) and then subjected to rat plasma analysis. A trend curve equation was obtained from the amount of the human FVII antigen remaining at the measured points of time. Finally, the half-lives of the respective test subjects were calculated by the equation ‘Half-life=LN(2)/Trend curve slope’. As listed in the following Table 6, it was revealed that the fusion proteins according to one exemplary embodiment of the present invention had the half-life 3 to 4 times higher than the non-fused FVII.

TABLE 6

| Kinds of fusion | |
|---|---|
| protein | Half-life (min) |
| FVII-GS1-Tf | 254.2 ± 19.1 |
| FVII-GS3-Tf | 227.4 ± 23.5 |
| FVII-GS1-T-Tf | 235.4 ± 27.4 |
| FVII-GS15-Tf | 257.0 ± 23.9 |
| FVII-Tf | 277.0 ± 24.5 |
| NovoSeven ® | 80.3 ± 27.4 |
| Wild-type FVII | 59.6 ± 2.9 |

The FVII-fusion proteins prepared in Examples 2 to 5 were expressed in CHO (VK2) cells that is derived from CHO cell line in which VKORC1 was stably expressed.

The expression vectors constructed in Examples 2 to 5 were purified using Endo-free plasmid maxi kit (Qiagen, #27104). β-Galactosidase was used as the control for transfection. CHO (VK2) cells were seeded in a 6-well plate at a concentration of 1.5×10⁶ cells/well. The cells were cultured for 24 hours in a-MEM (Lonza, #12-169F) supplemented with 10% FBS (Lonza, #14-501F), 1× HT (Invitrogen, #11067-030), 4 mM L-glutamine (Lonza, #17-605E), and 200 μg/mL of hygromycin (Invitrogen, #10687-010), and then the cells were transfected according to the supplier's manual using Lipofectamine 2000 (Invitrogen). After 4 hours of the transfection, the culture medium was replaced with a serum-free culture medium (OptiMEM), and 5 μg/mL of vitamin K was added thereto. After 48 hours of the culture, the culture medium was sampled, and stored at −70° C.

### EXAMPLE 9

### Isolation and Purification of FVII Fusion Protein (1)

<9-1> Expression of FVII Fusion Protein and Isolation/Concentration of Culture Medium

The expression vector including the FVII-GS1-T-Tf fusion protein was expressed in CHO cell line, as described above in Example <8-1>. The transfected cells were cultured at 37° C. for 3 to 5 days in a 5% CO₂ incubator, and the cell culture medium was filtered through a 0.22 μm filter (Corning) to remove cells and cell debris remaining in the culture medium. The filtered culture medium was concentrated 10 times by ultrafiltration using a tangential-flow membrane (Satorious, 30 kDa). Substances, such as the FVII fusion proteins, having a high molecular weight of 130 kDa do not pass through the filtration membrane, but substances having a low molecular weight, inorganic salts, or buffer components pass through the porous membrane. The concentrated culture medium was confirmed to have a yield of approximately 90%, compared to the activity (%) of the initial culture medium.

<9-2> Isolation and Purification of Fusion Protein by Affinity Chromatography

The culture medium concentrated in Example <9-1> was isolated and purified by affinity chromatography using an XK16/20 VIISelect column (GE Healthcare) packed with a VIISelect resin.

Specifically, prior to performing the chromatography, a VIISelect column was equilibrated with a buffer (pH 7.5) including 50 mM Tris and 150 mM sodium chloride. Thereafter, the column was loaded with the concentrated culture medium, and washed with a buffer (pH 7.5) including 50 mM Tris and 150 mM sodium chloride so as to remove the impurities in the column. After the washing, the FVII fusion protein was eluted using an aqueous 2.5 M sodium thiocyanate buffer (pH 7.0) as the elution buffer.

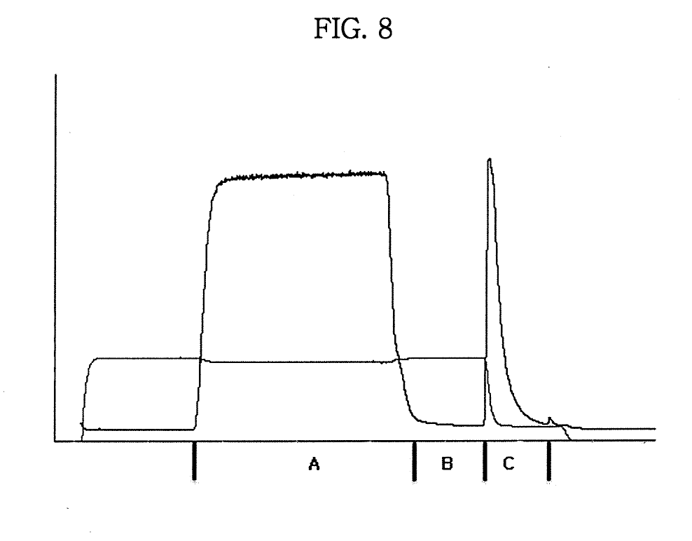

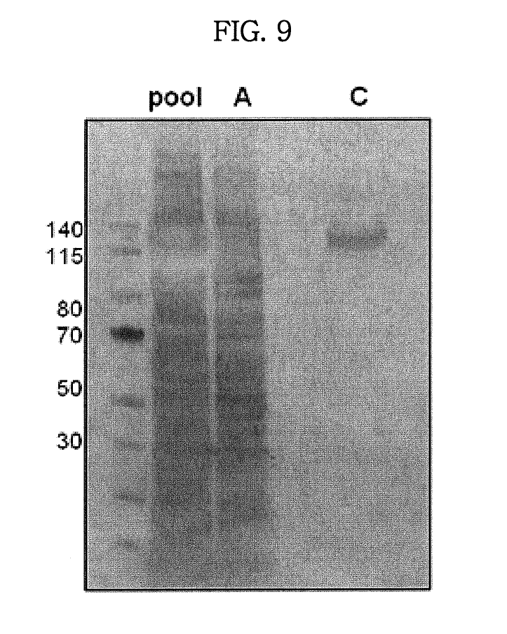

Using the chromatogram (GE healthcare, Equipment name: AKTA explorer) and SDS-PAGE, it was confirmed that the FVII fusion protein was present in the eluent. The results are shown in FIGS. 8 and 9. FIG. 8 shows the results analyzed by chromatogram in a purification procedure using VIISelect: A, B and C represent an injected culture medium, the impurities not bound to the resin, and the fusion protein eluate, respectively.

Also, FIG. 9 shows the results analyzed using Bio-rad ChemiDoc XRS program image analyzer after the fusion proteins are subjected to SDS-PAGE in a 4-12% Bis-Tris gel under the non-reducing conditions, followed by silver staining: A and C represent an injected culture medium, and the fusion protein eluate, respectively.

As seen from the results, the FVII fusion proteins having a purity of at least 95% or more were obtained in a yield at least 90% using the VIISelect column.

<9-3> Isolation and Purification of the Fusion Protein by Anion Exchange Chromatography

To isolate active fragments of the FVII fusion proteins, the eluent passed through the VIISelect column in Example <9-2> was subjected to anion exchange chromatography using a column (1.6×2.5 cm, Charge: 5 mL; GE Healthcare) packed with Q-sepharose FF gel, and the active fragment of the FVII fusion protein was further isolated and purified using a method in which protein binds to an amine group via net charge reaction.

Specifically, prior to performing the chromatography, the eluent obtained in Example <9-2> was concentrated and dialyzed with buffer (pH 8.0) including 20 mM Tris, 20 mM sodium chloride, and 5 mM calcium chloride.

Next, column was equilibrated at a flow rate of 5 mL/min using a buffer (pH 8.0) including 20 mM Tris, 20 mM sodium chloride, and 5 mM calcium chloride before the concentrated eluent was loaded onto the Q-sepharose FF column. After eluent was loaded onto the column, fragments of the FVII fusion protein was isolated by a gradient elution method using a buffer (pH 8.0) including 20 mM Tris, 5 mM calcium chloride, and 20 mM to 1 M sodium chloride.

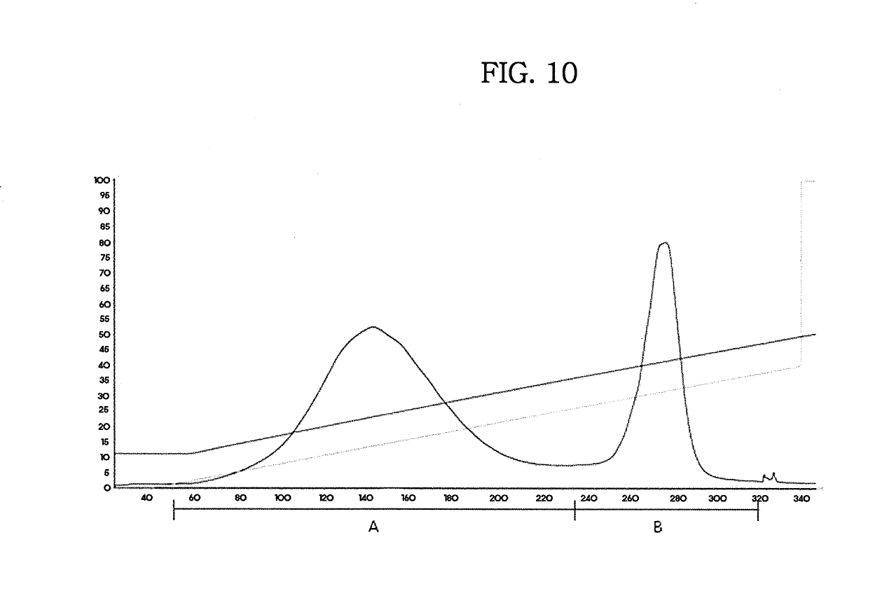

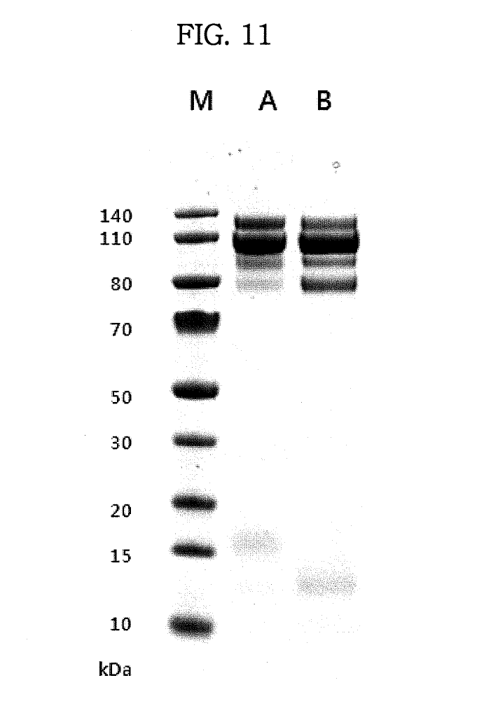

Using the chromatogram and SDS-PAGE, it was confirmed that the FVII fusion protein was present in the eluent. The results are shown in FIGS. 10 and 11.

FIG. 10 shows the results analyzed by chromatogram during a purification procedure using Q-sepharose FF gel: A and B represent an active fusion protein eluate and an inactive fusion protein eluate, respectively. From the chromatogram results, it could be seen that an active form of fragment A was eluted at a sodium chloride concentration of 86.64 mM.

Also, FIG. 11 shows the results analyzed using Bio-rad ChemiDoc XRS program image analyzer after the fusion proteins were subjected to SDS-PAGE in a 4-12% Bis-Tris gel under the reducing conditions, followed by Coomassie blue staining. In FIG. 11, A and B show the results analyzed by quantifying an active fusion protein eluate and an inactive form of the fragment at a concentration of 3 μg, respectively. As seen from the SDS-PAGE results, the active fragments (A) and the inactivated fragments (B) were able to be isolated using the purification method according to one exemplary embodiment of the present invention.

As seen from the results, the FVII fusion proteins having a purity of at least 98% or more were obtained in a yield at least 50% using the Q-sepharose FF column.

### COMPARATIVE EXAMPLE 1

### Isolation and Purification of FVII Fusion Protein by Affinity Chromatography Under Other Elution Conditions

The FVII fusion protein was isolated and purified by repeatedly performing the same procedure as in Example <9-2>, except that an elution buffer (pH 7.5) including 50 mM Tris, 1.5 M sodium chloride, and 50% propylene glycol was used instead of the elution buffer (pH 7.0) including 2.5 M sodium thiocyanate upon the isolation and purification using the affinity chromatography in Example <9-2>.

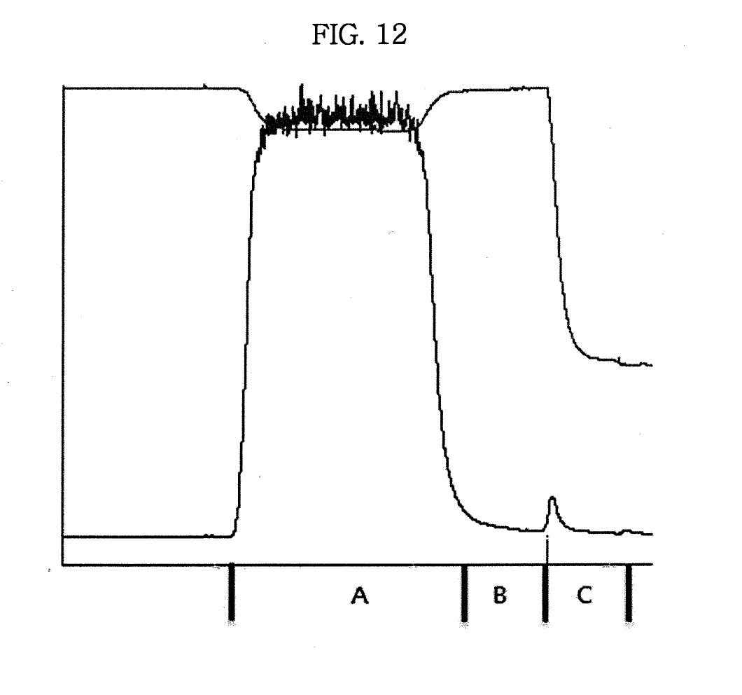

The eluents obtained by the method above were analyzed by chromatogram in the same manner as in Example <9-2> using chromatogram. The results are shown in FIG. 12. In the drawing, A, B, and C represent an injected culture medium, the impurities not bound to the resin, and the fusion protein eluate, respectively.

As seen in FIG. 12, the FVII fusion protein was recovered in a very low yield (5% or less) when the elution buffer (pH 7.5) including 50 mM Tris, 1.5 M sodium chloride, and 50% propylene glycol was used. By comparing the results of Example <9-2> to those of Comparative Example 1, the FVII fusion proteins were isolated and purified more effectively when the aqueous buffer (pH 7.0) including 2.5 M sodium thiocyanate was used in the affinity chromatography, instead of the elution buffer including 50 mM Tris, 1.5 M sodium chloride, and 50% propylene glycol.

### EXAMPLE 10

### Isolation and Purification of FVII Fusion Protein (2)

The procedure in Example 9 was repeatedly performed, except that mixed-mode chromatography to be described below was used instead of the affinity chromatography in Example <9-2>.

<10-1> Isolation and Purification of Fusion Protein by Mixed-Mode Chromatography

The culture medium concentrated in Example <9-1> was isolated and purified by mixed-mode chromatography using a column (Bio rad) packed with a ceramic fluoroapatite gel (Ca₁₀(PO₄)₆F₂).

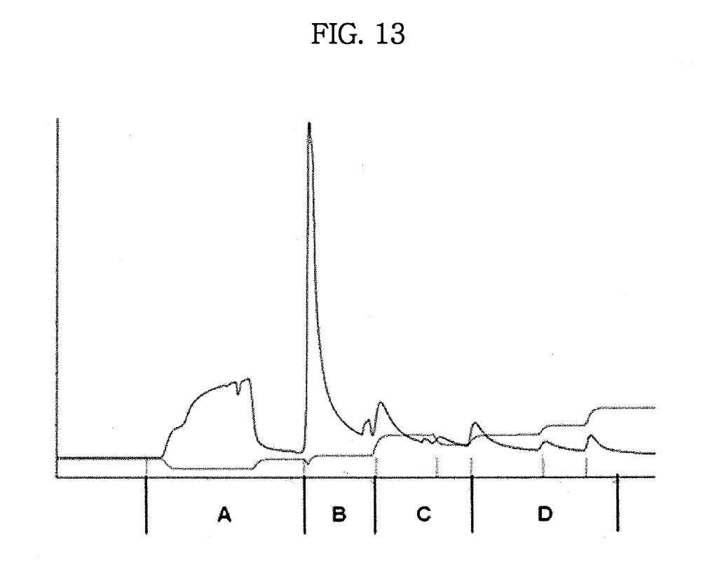

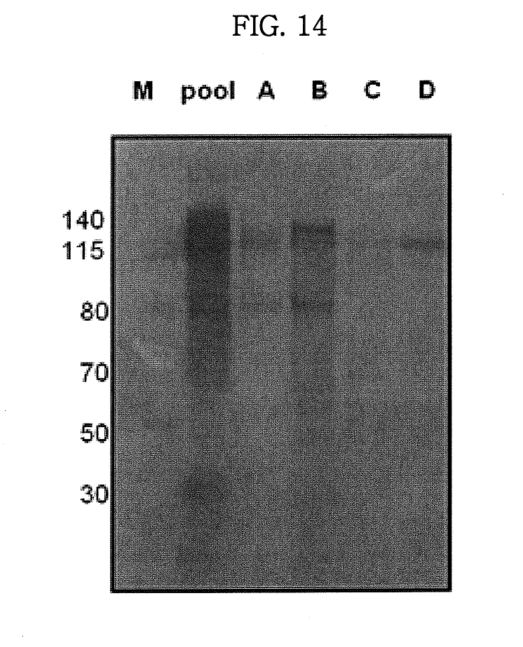

Specifically, prior to performing the chromatography, an XK16/20 ceramic fluoroapatite column was equilibrated with a buffer (pH 6.5) including 25 mM imidazole and 0.02% Tween 80. Thereafter, the concentrated culture medium obtained in Example <9-1> was loaded into the column, and the column was primarily washed with a buffer (pH 6.3) including 25 mM imidazole, 0.02% Tween 80, and 100 mM Na-Pi, and then secondarily washed with a buffer including 25 mM imidazole, 0.02% Tween 80, 100 mM Na-Pi, and 0.2 M sodium chloride (pH 6.3) so as to remove the impurities in the column and unwanted isoforms thereof. After the washing, the FVII fusion protein was eluted by a phosphate step gradient method using an elution buffer (pH 6.3) including 25 mM imidazole, 0.02% Tween 80, and 180-340 mM Na-Pi. Using the chromatogram and SDS-PAGE, it was confirmed that the FVII fusion protein was present in the eluent. The results are shown in FIGS. 13 and 14. FIG. 13 shows the results analyzed by chromatogram in a purification procedure using a ceramic fluoroapatite gel: A, B, C, and D represent an injected culture medium, primary washing eluate, secondary washing eluate, and a fusion protein eluate, respectively. Also, FIG. 14 shows the results analyzed using Bio-rad ChemiDoc XRS program image analyzer after the fusion proteins are subjected to SDS-PAGE in a 4-12% Bis-Tris gel under the non-reducing conditions, followed by silver staining: A, B, C, and D represent an injected culture medium, a primary washing eluate, secondary washing eluate, and a fusion protein eluate, respectively.

As seen from the results, the FVII fusion proteins having a purity of at least 90% or more were obtained in a yield at least 60% using the mixed-mode ceramic fluoroapatite column.

<10-2> Isolation and Purification of the Fusion Protein by Anion Exchange Chromatography

Next, the eluent passed through the ceramic fluoroapatite gel was isolated and purified by the anion exchange chromatography as described in Example <9-3>. The FVII fusion proteins having a purity of at least 98% or more were able to be obtained in a yield at least 50% through the isolation and purification.

### COMPARATIVE EXAMPLE 2

### Isolation and Purification of FVII Fusion Protein Using Ceramic Hydroxyapatite Column

The procedure in Example <10-1> was repeatedly performed, except that a ceramic hydroxyapatite column to be described below was used instead of the ceramic fluoroapatite column upon the isolation and purification using the mixed-mode chromatography in Example <10-1>.

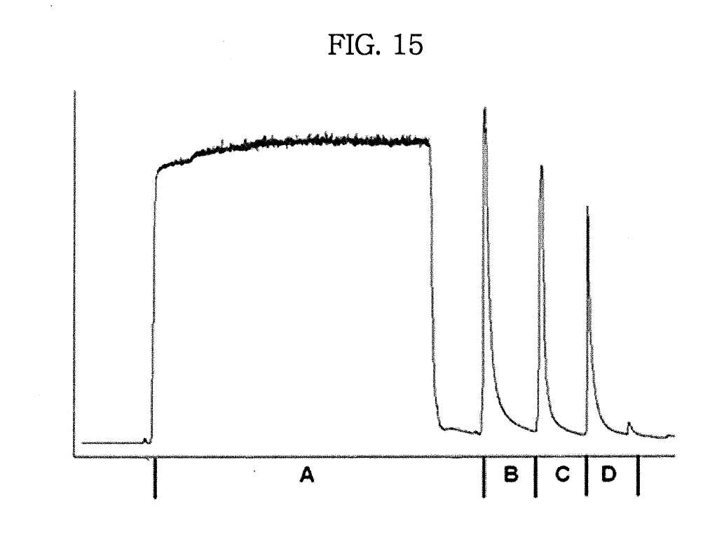

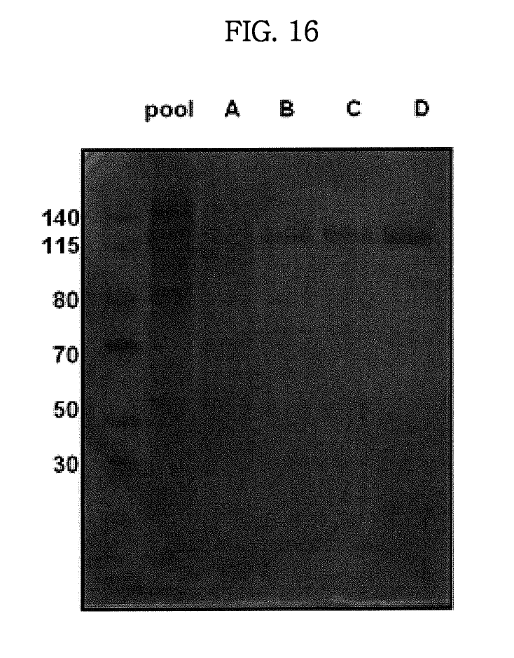

Specifically, prior to performing the chromatography, an XK16/20 ceramic hydroxyapatite column (Bio-rad) was equilibrated with a buffer (pH 6.5) including 25 mM imidazole, 100 mM sodium chloride, and 0.02% Tween 80. Thereafter, the concentrated culture medium obtained in Example <9-1> was loaded into the column, and the column was primarily washed with a buffer (pH 6.3) including 25 mM imidazole, 0.02% Tween 80, and 100 mM Na-Pi, and then secondarily washed with a buffer (pH 6.3) including 25 mM imidazole, 0.02% Tween 80, 100 mM Na-Pi, and 0.6 M sodium chloride so as to remove the impurities in the column and unwanted isoforms thereof. After the washing, the FVII fusion protein was eluted by a phosphate step gradient method using an elution buffer (pH 6.3) including 25 mM imidazole, 0.02% Tween 80, and 180-340 mM Na-Pi. Using the chromatogram and SDS-PAGE, it was confirmed that the FVII fusion protein was present in the eluent. The results are shown in FIGS. 15 and 16. FIG. 15 shows the results analyzed by chromatogram in a purification procedure using a ceramic hydroxyapatite gel: A, B, C, and D represent injected culture medium, primary washing eluate, secondary washing eluate, and the fusion protein eluate, respectively. Also, FIG. 16 shows the results analyzed using Bio-rad ChemiDoc XRS program image analyzer after the fusion proteins are subjected to SDS-PAGE in a 4-12% Bis-Tris gel under the non-reducing conditions, followed by silver staining: A, B, C, and D represent injected culture medium, primary washing eluate, secondary washing eluate, and the fusion protein eluate, respectively.

As seen from the results, the results obtained by the ceramic hydroxyapatite column showed that the FVII fusion proteins having a purity of at least 90% or more were obtained in a yield at least 52%. As seen from the results, that the yield was reduced, compared to the results obtained through the ceramic fluoroapatite column used in Example <10-1>.

What is claimed is:

1. A method of isolating and purifying a fusion protein comprising factor VII, the method comprising:
1) isolating and purifying a fusion protein comprising factor VII expressed in animal cells by affinity chromatography using a resin having a structure represented by Formula 1 as a stationary phase or mixed-mode chromatography using a ceramic fluoroapatite gel (Ca₁₀(PO₄)₆F₂) as a stationary phase; and
2) further isolating and purifying the fusion protein by anion exchange chromatography using Q-sepharose FF gel as a stationary phase: 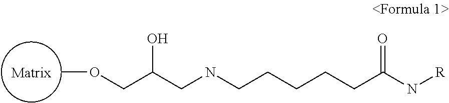
wherein the matrix is cross-linked agarose, and R is a factor VII binding protein serving as a ligand. 15

2. The method of claim 1, wherein the animal cells are selected from the group consisting of CHO cells, BHK21 cells, HEK293 cells, and Hep G2 cells.

3. The method of claim 1, wherein the fusion protein comprising the factor VII is a protein in which the factor VII is fused to transferrin.

4. The method of claim 3, wherein the fusion protein comprises a linker between the factor VII and the transferrin.

5. The method of claim 4, wherein the linker is a peptide having an amino acid sequence set forth in one of SEQ ID NOs: 3 to 12.

6. The method of claim 1, wherein the affinity chromatography in step 1 uses an aqueous buffer (pH 5.0 to 8.0) containing sodium thiocyanate as an elution buffer.

7. The method of claim 1, wherein the mixed-mode chromatography in step 1 uses an aqueous buffer (pH 5.0 to 8.0) comprising sodium phosphate and sodium chloride.

8. The method of claim 1, wherein the anion exchange chromatography in step 2 uses an aqueous buffer (pH 5.0 to 8.0), which comprises 10 mM to 50 mM Tris, 1 mM to 10 mM calcium chloride, and 50 mM to 150 mM sodium chloride, as an elution buffer.


SEQUENCE LISTING (53 sequences)

SEQ ID NO: 1 - protein, 444 aa, Homo sapiens
  PEPTIDE (1)..(444): Factor VII protein
MVSQALRLLCLLLGLQGCLAAVFVTQEEAHGVLHRRRRANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFP

SEQ ID NO: 2 - protein, 679 aa, Homo sapiens
  PEPTIDE (1)..(679): Transferrin protein
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 3 - protein, 5 aa, Artificial Sequence
  Linker GS-1
GGGGS

SEQ ID NO: 4 - protein, 15 aa, Artificial Sequence
  Linker GS-3
GGGGSGGGGSGGGGS

SEQ ID NO: 5 - protein, 25 aa, Artificial Sequence
  Linker GS-5
GGGGSGGGGSGGGGSGGGGSGGGGS

SEQ ID NO: 6 - protein, 35 aa, Artificial Sequence
  Linker GS-7
GGGGSGGGGSGGGGSGGGGSGGGGSGGGGSGGGGS

SEQ ID NO: 7 - protein, 45 aa, Artificial Sequence
  Linker GS-9
GGGGSGGGGSGGGGSGGGGSGGGGSGGGGSGGGGSGGGGSGGGGS

SEQ ID NO: 8 - protein, 55 aa, Artificial Sequence
  Linker GS-11
GGGGSGGGGSGGGGSGGGGSGGGGSGGGGSGGGGSGGGGSGGGGSGGGGSGGGGS

SEQ ID NO: 9 - protein, 65 aa, Artificial Sequence
  Linker GS-13
GGGGSGGGGSGGGGSGGGGSGGGGSGGGGSGGGGSGGGGSGGGGSGGGGSGGGGSGGGGSGGGGS

SEQ ID NO: 10 - protein, 75 aa, Artificial Sequence
  Linker GS-15
GGGGSGGGGSGGGGSGGGGSGGGGSGGGGSGGGGSGGGGSGGGGSGGGGSGGGGSGGGGSGGGGSGGGGSGGGGS

SEQ ID NO: 11 - protein, 23 aa, Artificial Sequence
  Linker Helix
GAEAAAKEAAAKEAAAKEAAAKA

SEQ ID NO: 12 - protein, 15 aa, Artificial Sequence
  Linker GS1-T
GGGGSLVPRGSGGGS

SEQ ID NO: 13 - DNA, 3378 bp, Artificial Sequence
  gene sequence encoding FVII-Tf protein
atggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttcccaccggtgtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacctggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcaggagccttcaagtgtctgaaggatggtgctggggatgtggcctttgtcaagcactcgactatatttgagaacttggcaaacaaggctgacagggaccagtatgagctgctttgcctggacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtcccccccaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaa

SEQ ID NO: 14 - DNA, 3387 bp, Artificial Sequence
  gene sequence encoding FVII-GS1-Tf protein
atggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttcccggtggaggcggatccgtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacctggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcaggagccttcaagtgtctgaaggatggtgctggggatgtggcctttgtcaagcactcgactatatttgagaacttggcaaacaaggctgacagggaccagtatgagctgctttgcctggacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtcccccccaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaa

SEQ ID NO: 15 - DNA, 3426 bp, Artificial Sequence
  gene sequence encoding FVII-GS3-Tf protein
atggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttcccaccggtggaggcggttcaggcggaggtggctctggcggtggcggatccaccggtgtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacctggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcaggagccttcaagtgtctgaaggatggtgctggggatgtggcctttgtcaagcactcgactatatttgagaacttggcaaacaaggctgacagggaccagtatgagctgctttgcctggacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtcccccccaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaa

SEQ ID NO: 16 - DNA, 3456 bp, Artificial Sequence
  gene sequence encoding FVII-GS5-Tf protein
atggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttcccaccggtggaggcggttcaggcggaggtggctctggcggtggcggatctggcggaggtggctctggcggtggcggatccaccggtgtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacctggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcaggagccttcaagtgtctgaaggatggtgctggggatgtggcctttgtcaagcactcgactatatttgagaacttggcaaacaaggctgacagggaccagtatgagctgctttgcctggacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtcccccccaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaa

SEQ ID NO: 17 - DNA, 3486 bp, Artificial Sequence
  gene sequence encoding FVII-GS7-Tf protein
atggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttcccaccggtggaggcggttcaggcggaggtggctctggcggtggcggatctggcggaggtggctctggcggtggcggatctggcggaggtggctctggcggtggcggatccaccggtgtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacctggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcaggagccttcaagtgtctgaaggatggtgctggggatgtggcctttgtcaagcactcgactatatttgagaacttggcaaacaaggctgacagggaccagtatgagctgctttgcctggacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtcccccccaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaa

SEQ ID NO: 18 - DNA, 3516 bp, Artificial Sequence
  gene sequence encoding FVII-GS9-Tf protein
atggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttcccaccggtggaggcggttcaggcggaggtggctctggcggtggcggatctggcggaggtggctctggcggtggcggatctggcggaggtggctctggcggtggcggatctggcggaggtggctctggcggtggcggatccaccggtgtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacctggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcaggagccttcaagtgtctgaaggatggtgctggggatgtggcctttgtcaagcactcgactatatttgagaacttggcaaacaaggctgacagggaccagtatgagctgctttgcctggacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtcccccccaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaa

SEQ ID NO: 19 - DNA, 3546 bp, Artificial Sequence
  gene sequence encoding FVII-GS11-Tf protein
atggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttcccaccggtggaggcggttcaggcggaggtggctctggcggtggcggatctggcggaggtggctctggcggtggcggatctggcggaggtggctctggcggtggcggatctggcggaggtggctctggcggtggcggatctggcggaggtggctctggcggtggcggatccaccggtgtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacctggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcaggagccttcaagtgtctgaaggatggtgctggggatgtggcctttgtcaagcactcgactatatttgagaacttggcaaacaaggctgacagggaccagtatgagctgctttgcctggacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtcccccccaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaa

SEQ ID NO: 20 - DNA, 3576 bp, Artificial Sequence
  gene sequence encoding FVII-GS13-Tf protein
atggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttcccaccggtggaggcggttcaggcggaggtggctctggcggtggcggatctggcggaggtggctctggcggtggcggatctggcggaggtggctctggcggtggcggatctggcggaggtggctctggcggtggcggatctggcggaggtggctctggcggtggcggatctggcggaggtggctctggcggtggcggatccaccggtgtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacctggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcaggagccttcaagtgtctgaaggatggtgctggggatgtggcctttgtcaagcactcgactatatttgagaacttggcaaacaaggctgacagggaccagtatgagctgctttgcctggacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtcccccccaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaa

SEQ ID NO: 21 - DNA, 3606 bp, Artificial Sequence
  gene sequence encoding FVII-GS15-Tf protein
atggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttcccaccggtggaggcggttcaggcggaggtggctctggcggtggcggatctggcggaggtggctctggcggtggcggatctggcggaggtggctctggcggtggcggatctggcggaggtggctctggcggtggcggatctggcggaggtggctctggcggtggcggatctggcggaggtggctctggcggtggcggatctggcggaggtggctctggcggtggcggatccaccggtgtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacctggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcaggagccttcaagtgtctgaaggatggtgctggggatgtggcctttgtcaagcactcgactatatttgagaacttggcaaacaaggctgacagggaccagtatgagctgctttgcctggacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtcccccccaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaa

SEQ ID NO: 22 - DNA, 3450 bp, Artificial Sequence
  gene sequence encoding FVII-Helix-Tf protein
atggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttcccaccggtgctgaagctgcagccaaagaagctgcagccaaagaggccgcagctaaggaagccgcagcaaaagctaccggtgtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacctggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcaggagccttcaagtgtctgaaggatggtgctggggatgtggcctttgtcaagcactcgactatatttgagaacttggcaaacaaggctgacagggaccagtatgagctgctttgcctggacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtcccccccaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaa

SEQ ID NO: 23 - DNA, 3426 bp, Artificial Sequence
  gene sequence encoding FVII-GS1-T-Tf protein
atggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttcccaccggtggaggcggatccctggtgccgcgcggcagcggaggcggttcaaccggtgtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacctggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcaggagccttcaagtgtctgaaggatggtgctggggatgtggcctttgtcaagcactcgactatatttgagaacttggcaaacaaggctgacagggaccagtatgagctgctttgcctggacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtcccccccaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaa

SEQ ID NO: 24 - DNA, 3417 bp, Artificial Sequence
  gene sequence encoding FVII-GS1-T-Tf(M3) protein
atggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttcccggtggaggcggatccctggtgccgcgcggcagcggaggcggttcagtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacctggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcaggagccttcaagtgtctgaaggatggtgctggggatgtggcctttgtcaagcactcgactatatttgagaacttggcaaacaaggctgacagggaccagtatgagctgctttgcctggacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtcccccccaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaa

SEQ ID NO: 25 - DNA, 25 bp, Artificial Sequence
  FVII-F primer
aggggcagcactgcagagatttcat

SEQ ID NO: 26 - DNA, 25 bp, Artificial Sequence
  FVII-R primer
tatgggatttggtgccaggacagtt

SEQ ID NO: 27 - DNA, 31 bp, Artificial Sequence
  FVII-S1 primer
aattgctagcatggtctcccaggccctcagg

SEQ ID NO: 28 - DNA, 31 bp, Artificial Sequence
  FVII-AS1 primer
aattaccggtgggaaatggggctcgcaggag

SEQ ID NO: 29 - DNA, 33 bp, Artificial Sequence
  Tf-S1 primer
atataccggtgtccctgataaaactgtgagatg

SEQ ID NO: 30 - DNA, 34 bp, Artificial Sequence
  Tf-AS1 primer
aattctcgagttaaggtctacggaaagtgcaggc

SEQ ID NO: 31 - DNA, 36 bp, Artificial Sequence
  GS-FV-AS1 primer
ggatccgcctccaccgggaaatggggctcgcaggag

SEQ ID NO: 32 - DNA, 40 bp, Artificial Sequence
  GS-Tf-S1 primer
ggtggaggcggatccgtccctgataaaactgtgagatggt

SEQ ID NO: 33 - DNA, 48 bp, Artificial Sequence
  GS3-S primer
ccggtggaggcggttcaggcggaggtggctctggcggtggcggatcca

SEQ ID NO: 34 - DNA, 48 bp, Artificial Sequence
  GS3-AS primer
acctccgccaagtccgcctccaccgagaccgccaccgcctaggtggcc

SEQ ID NO: 35 - DNA, 50 bp, Artificial Sequence
  GS-2 linker unit
tattagatctggcggaggtggctctggcggtggcggatccaccggtatta

SEQ ID NO: 36 - DNA, 60 bp, Artificial Sequence
  dsGS1-T linker
aattaccggtggaggcggatccctggtgccgcgcggcagcggaggcggttcaaccggtat

SEQ ID NO: 37 - DNA, 58 bp, Artificial Sequence
  Helix linker S primer
aattaccggtgctgaagctgcagccaaagaagctgcagccaaagaggccgcagctaag

SEQ ID NO: 38 - DNA, 46 bp, Artificial Sequence
  Helix linker AS primer
ttataccggtagcttttgctgcggcttccttagctgcggcctcttt

SEQ ID NO: 39 - DNA, 31 bp, Artificial Sequence
  Albumin-S primer
gtgggatccgatgcacacaagagtgaggttg

SEQ ID NO: 40 - DNA, 35 bp, Artificial Sequence
  Albumin-AS primer
cacggatccctataagcctaaggcagcttgacttg

SEQ ID NO: 41 - DNA, 55 bp, Artificial Sequence
  oligonucleotide 1
gtgctcgagcgggggatctggcgggtctggaggctctggagggtcgggaggctct

SEQ ID NO: 42 - DNA, 74 bp, Artificial Sequence
  oligonucleotide 2
caagggcccttatcaggatcccgaccctccagacccgccagatcccccagagcctccagagcctcccgaccctc

SEQ ID NO: 43 - DNA, 33 bp, Artificial Sequence
  mut FVII(XhoI)-S primer
gagccccatttccctcgagcccagcagccctgg

SEQ ID NO: 44 - DNA, 33 bp, Artificial Sequence
  mut FVII(XhoI)-AS primer
ccagggctgctgggctcgagggaaatggggctc

SEQ ID NO: 45 - protein, 31 aa, Artificial Sequence
  GS linker (EP1816201)
SSGGSGGSGGSGGSGGSGGSGGSGGSGGSGS

SEQ ID NO: 46 - DNA, 25 bp, Artificial Sequence
  Primer Nhe-Tf
aattgctagcatgaggctcgccgtg

SEQ ID NO: 47 - DNA, 28 bp, Artificial Sequence
  Primer Tf-Age
aattaccggtaggtctacggaaagtgca

SEQ ID NO: 48 - DNA, 25 bp, Artificial Sequence
  Primer Age-VII
aattaccggtgccaacgcgttcctg

SEQ ID NO: 49 - DNA, 28 bp, Artificial Sequence
  Primer VII-Xho
aattctcgagttagggaaatggggctcg

SEQ ID NO: 50 - DNA, 32 bp, Artificial Sequence
  Primer TG del-S
cagcggaggcggttcagtccctgataaaactg

SEQ ID NO: 51 - DNA, 32 bp, Artificial Sequence
  Primer TG del-AS
cagttttatcagggactgaaccgcctccgctg

SEQ ID NO: 52 - DNA, 29 bp, Artificial Sequence
  Primer T del-S
cgagccccatttcccggtggaggcggatc

SEQ ID NO: 53 - DNA, 29 bp, Artificial Sequence
  Primer T del-AS
gatccgcctccaccgggaaatggggctcg